SEQUENCE LISTING (26 sequences)

SEQ ID NO: 1 - DNA, 15186 bp, Newcastle disease virus
accaaacagagaatccgtgagctacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcatccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaggggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggcatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 2 - DNA, 15612 bp, Newcastle disease virus
accaaacagagaatccgtgagctacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcatccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaggggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatgaacgcactccaggaggacacaccacctggaccctctaccgtgttccggccacccaccagcagccggcccctggagacaccccactgccgggagatcagaatcggcatcgccggcatcacaatcaccctgagcctgtgcggatgtgcaaatgccagggcccccacactgcgctccgccaccgccgacaactctgagagcaccggctttaagaatgtgcctgacctgcggacagatcagccaaagcctccatccaagaagaggtcttgtgatcctagcgagtaccgcgtgtccgagctgaaggagtctctgatcaccacaacccccagcaggcctcgcaccgcaaaaaggcgtataagactgtaagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggcatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 3 - DNA, 16302 bp, Newcastle disease virus
accaaacagagaatccgtgagctacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcatccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaggggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaatacaattaacgatctttatagccaccatgacgatgaaaatgatggtacatatatatttcgtatcattattgttattgctattccacagttacgccatagacatcgaaaatgaaatcacagaattcttcaataaaatgagagatactctaccagctaaagactctaaatggttgaatccagcatgtatgttcggaggcacaatgaatgatatagccgctctaggagagccattcagcgcaaagtgtcctcctattgaagacagtcttttatcgcacagatataaagactatgtggttaaatgggaaaggctagaaaaaaatagacggcgacaggtttctaataaacgtgttaaacatggtgatttatggatagccaactatacatctaaattcagtaaccgtaggtatttgtgcaccgtaactacaaagaatggtgactgtgttcagggtatagttagatctcatattagaaaacctccttcatgcattccaaaaacatatgaactaggtactcatgataagtatggcatagacttatactgtggaattctttacgcaaaacattataataatataacttggtataaagataataaggaaattaatatcgacgacattaagtattcacaaacgggaaaggaattaattattcataatccagagttagaagatagcggaagatacgactgttacgttcattacgacgacgttagaatcaagaatgatatcgtagtatcaagatgtaaaatacttacggttataccgtcacaagaccacaggtttaaactaatactagatccaaaaatcaacgtaacgataggagaacctgccaatataacatgcactgctgtgtcaacgtcattattgattgacgatgtactgattgaatgggaaaatccatccggatggcttataggattcgattttgatgtatactctgttttaactagtagaggcggtattaccgaggcgaccttgtactttgaaaatgttactgaagaatatataggtaatacatataaatgtcgtggacacaactattattttgaaaaaacccttacaactacagtagtattggagtaagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggcatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 4 - DNA, 17412 bp, Newcastle disease virus
accaaacagagaatccgtgagctacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcatccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaggggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagttcggcctgtcctgggtgttcctggtggccctgtttagaggcgtgcagtgccaggtgcagctggtggagagcggaggaggagtggtgcagccaggccggagcctgcgcctggactgtaaggccagcggcatcacctttagcaactccggaatgcactgggtgaggcaggcaccaggcaagggcctggagtgggtggccgtgatctggtacgacggctctaagcggtactatgccgatagcgtgaagggccggttcacaatctccagagacaactctaagaataccctgttcctccagatgaacagcctgagggccgaggatacagccgtgtactattgcgccaccaatgacgattactggggccagggcacactggtgaccgtgagcagcgccagcaccaagggaccaagcgtgttcccactggcaccttgctctcgcagcacatccgagtctaccgccgccctgggatgtctggtgaaggattatttccccgagcctgtgaccgtgagctggaacagcggcgccctgacatccggagtgcacacctttcccgccgtgctccagagcagcggcctgtacagcctgtcctctgtggtgacagtgcccagctcctctctgggcaccaagacatatacctgcaacgtggaccacaagcctagcaataccaaggtggataagcgggtggagtccaagtacggaccaccttgcccaccatgtccagcaccagagttcctgggaggaccaagcgtgttcctgtttcctccaaagcccaaggacaccctgatgatctccagaacaccagaggtgacctgcgtggtggtggacgtgtctcaggaggaccccgaggtgcagttcaactggtatgtggatggcgtggaggtgcacaatgccaagaccaagcccagggaggagcagtttaactccacataccgcgtggtgtctgtgctgaccgtgctgcaccaggactggctgaacggcaaggagtataagtgcaaggtgtccaataagggcctgcctagctccatcgagaagacaatcagcaaggcaaagggacagccaagggagccacaggtgtacaccctgcccccttctcaggaggagatgacaaagaaccaggtgagcctgacctgtctggtgaagggcttctatccttccgacatcgccgtggagtgggagtctaatggccagccagagaacaattacaagaccacaccacccgtgctggactctgatggcagcttctttctgtattccaggctgacagtggataagtctcgctggcaggagggcaacgtgtttagctgctccgtgatgcacgaggccctgcacaatcactacacccagaagtctctgagcctgtccctgggcaagcggagaaagaggggcagcggagagggaaggggctccctgctgacatgtggcgacgtggaggagaatcctggcccaatgaagtatctgctgcccaccgccgccgccggcctgctgctgctggcagcacagcctgctatggccgagatcgtgctgacacagtccccagccaccctgtctctgagccctggagagagggccaccctgagctgtagagcatcccagagcgtgagcagctacctggcctggtatcagcagaagcctggacaggcaccaaggctgctgatctacgatgcaagcaacagggcaacaggcatccccgcaagattcagcggctccggctctggcaccgactttacactgaccatctcctctctggagcctgaggatttcgccgtgtactattgccagcagagcagcaattggccacggacatttggccagggcaccaaggtggagatcaagagaacagtggccgcccctagcgtgttcatctttcctccatccgacgagcagctgaagagcggcaccgcatccgtggtgtgcctgctgaacaatttctacccacgggaggccaaggtgcagtggaaggtggataacgccctccagtccggcaattctcaggagagcgtgacagagcaggactccaaggattctacctatagcctgtctagcacactgaccctgtccaaggccgactacgagaagcacaaggtgtatgcctgcgaggtgacacaccagggcctgtcctctcccgtgaccaagagctttaacagaggcgagtgctgataagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggcatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 5 - DNA, 36 bp, Newcastle disease virus
acgactcactataggaccaaacagagaatccgtgag

SEQ ID NO: 6 - DNA, 35 bp, Newcastle disease virus
ccgggaagatccagggcactcttcttgcatgttac

SEQ ID NO: 7 - DNA, 35 bp, Newcastle disease virus
gggcctgcctcactatggtggtaacatgcaagaag

SEQ ID NO: 8 - DNA, 35 bp, Newcastle disease virus
tgcatgttaccaccaatgtgtcattgtatcgcttg

SEQ ID NO: 9 - DNA, 37 bp, Newcastle disease virus
caagaagggagatacgtaatatacaagcgatacaatg

SEQ ID NO: 10 - DNA, 35 bp, Newcastle disease virus
tcgcttgtatattacttgttgtagcaaagagcacc

SEQ ID NO: 11 - DNA, 38 bp, Newcastle disease virus
ggcctggatcttcccattatgctgtctgtatacggtgc

SEQ ID NO: 12 - DNA, 38 bp, Newcastle disease virus
atgccatgccgacccaccaaacaaagacttggtgaatg

SEQ ID NO: 13 - DNA, 19 bp, Newcastle disease virus
accaaacagagaatccgtg

SEQ ID NO: 14 - DNA, 20 bp, Newcastle disease virus
agtcttcagtcatggacagc

SEQ ID NO: 15 - DNA, 35 bp, Newcastle disease virus
ctctagagtcgacccttctgccaacatgtcttccg

SEQ ID NO: 16 - DNA, 35 bp, Newcastle disease virus
gggaagcggccgcccgtcggtcagtatccccagtc

SEQ ID NO: 17 - DNA, 37 bp, Newcastle disease virus
ctctagagtcgaccccagagtgaagatggccaccttc

SEQ ID NO: 18 - DNA, 35 bp, Newcastle disease virus
gggaagcggccgcccagtgatcagccattcagcgc

SEQ ID NO: 19 - DNA, 35 bp, Newcastle disease virus
ctctagagtcgacccgggtaggacatggcgggctc

SEQ ID NO: 20 - DNA, 37 bp, Newcastle disease virus
gggaagcggccgccctgcctttaagagtcacagttac

SEQ ID NO: 21 - DNA, 191 bp, Newcastle disease virus
aggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcatgtcattcaccaaatctttgtttggt

SEQ ID NO: 22 - DNA, 176 bp, Newcastle disease virus
aggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcatgtcattcacc

SEQ ID NO: 23 - DNA, 140 bp, Newcastle disease virus
acttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 24 - DNA, 140 bp, Newcastle disease virus; identical to SEQ ID NO: 23

SEQ ID NO: 25 - DNA, 113 bp, Newcastle disease virus
acttcattataccatattagaaaaaaattgaattccgaccctttaagactcgtattcggattcaaataattatctcagaaaaaaagtgcacgtagttgttcttgattatagtc

SEQ ID NO: 26 - DNA, 25 bp, Newcastle disease virus
gtcattcaccaagtctttgtttggt

(19) United States
(12) Patent Application Publication
(10) Pub. No.: US 2021/0169956 A1
(43) Pub. Date: Jun. 10, 2021

(54) INCREASED ACTIVITY OF ONCOLYTIC NEWCASTLE DISEASE VIRUS

(71) Applicants: Gerhard Willi Michael Noss, Wasserburg (DE); Arno Thaller, Markt Berolzheim (DE); RAPOYERAPEH B.H. LTD, Alon Shvut (IL)

(72) Inventors: Gerhard Willi Michael NOSS, Wasserburg (DE); Arno THALLER, Markt Berolzheim (DE); Olav de LEEUW, Almere (NL); Ben PEETERS, Lelystad (NL)

(73) Assignee: Rapo Yerapeh B.H. Ltd, Alon Shvut (IL)

(21) Appl. No.: 17/046,462

(22) PCT Filed: Apr. 4, 2019
(86) PCT No.: PCT/EP2019/058565

§ 371 Date: Oct. 9, 2020

(30) Foreign Application Priority Data
Apr. 9, 2018 (EP) 18166400.4

(51) Int. Cl. A61K 35/768; C12N 7/00; C07K 14/005; A61P 35/00

(52) U.S. Cl. CPC A61K 35/768; C12N 7/00; C07K 14/005; C12N 2760/18122; C12N 2760/18121; C12N 2760/18132; A61P 35/00

(57) ABSTRACT

The invention relates to Newcastle Disease Vims (NDV), an avian paramyxovirus, which has been demonstrated to possess significant oncolytic activity against mammalian cancers. The invention provides the elucidation of the mechanisms of NDV-mediated oncolysis as well as the development of novel oncolytic viruses through the use of genetic engineering. The invention also provides a nucleic acid encoding a reverse genetically engineered (rg-)NDV having a mutation in the HN gene, said mutation allowing replication of said rgNDV in a cancer cell to a higher level than replication of an otherwise identical rgNDV not having said mutation in the HN gene.

This invention relates to Newcastle Disease Virus (NDV), an avian paramyxovirus, which has been demonstrated to possess significant oncolytic activity against mammalian cancers, especially selected from the specific cancers as described herein and/or mentioned in the claims. The invention provides the elucidation of the mechanisms of NDV-mediated oncolysis as well as the development of novel and improved oncolytic agents through the use of genetic engineering.

### BACKGROUND

Oncolytic viruses, which are well known to inherently replicate selectively within tumor cells and kill tumor cells while leaving non-tumor cells unharmed, provide an attractive new tool for cancer immunotherapy. Typically, an oncolytic virus is defined as a virus for use in oncological treatment, preferably in treatment of human subjects in need thereof. A number of RNA viruses, including NDV, reovirus, measles virus, and vesicular stomatitis virus (VSV), are members of this novel class of viruses being exploited as potential oncolytic agents.

NDV is an intrinsically tumor-specific virus, which is under investigation as a clinical oncolytic immunotherapy agent, and whose safety margin is a function of its inability to replicate and kill normal human cells. NDV derives its name from the site of the original outbreak in chickens at a farm near Newcastle-upon-Tyne in England in 1926. It is an economically important pathogen in multiple avian species and it is endemic in many countries. NDV is a member of the Avulavirus genus in the Paramyxoviridae family. Several clinical trials have reported NDV to be a safe and therapeutically useful agent for cancer therapy (Cassel et al., 1965, Cancer 1:863-888; Steiner et al., 2004, J. Clin. Oncol. 22:4272-4281; Schulze et al., 2009, Cancer Immunol. Immunother. 58:61-69; Csatary et al., 1999, Anticancer Res. 19:635-638; Pecora et al., 2002, J. Clin. Oncol. 20:2251-2266; Lorence et al., 2003, Curr. Opin. Mol. Ther. 5:618-624; Freeman et al., 2006, Mol. Ther. 13:221-228); however, there remains a clear need for improvement in therapeutic outcome, in particular to enhancing its killing effect without affecting its safety toward normal cells. NDV is a non-segmented, negative-strand RNA virus of the Paramyxoviridae family, whose natural host range is limited to avian species; however, it is known to enter cells by binding to sialic acid residues present on a wide range of human and rodent cancer cells. NDV has been shown to selectively replicate in and destroy tumor cells, while sparing normal cells, an oncolytic property believed to be based in part on defective antiviral responses in tumor cells. Normal cells, which are competent in launching an efficient antiviral response rapidly after infection, are able to inhibit viral replication before viral-mediated cell damage can be initiated. The sensitivity of NDV to interferon (IFN), coupled with defective IFN signaling pathways in tumor cells, provides one plausible mechanism whereby NDV can replicate exclusively within neoplastic tissue. The selective replication of NDV in tumor cells may also be caused by several other mechanisms, including defects in activation of anti-viral signaling pathways, defects in apoptotic pathways, and activation of Ras signaling and/or expression of Rac1 (Schirrmacher, 2015, Expert Opin. Biol. Ther. 15:17 57-71).

Typically, an oncolytic NDV strain is defined as an NDV for use in oncological immunotherapy, preferably in treatment of human subjects in need thereof. Multiple preclinical model studies have shown significant anti-tumor activity of natural and recombinant oncolytic NDV after varying treatment modalities. Promising results were noted in preclinical models of glioblastoma multiforme, anaplastic astrocytoma, leukemia, lymphoma, melanoma, neuroblastoma, osteosarcoma, rhabdomyosarcoma, Ewing's sarcoma, fibrosarcoma, pheochromocytoma, colon carcinoma, lung carcinoma, prostate carcinoma, breast carcinoma, ovary carcinoma, gastric carcinoma, mesothelioma, renal cell carcinoma, and head & neck carcinoma. One typically useful oncolytic NDV that has been extensively explored for use of treatment in humans, and for which an advantageous safety and efficacy profile has been established in human trials, is the strain named MTH-68/H (Csatary L K, et al., J Neurooncol. 2004 March-April; 67(1-2):83-93). MTH-68/H therapy has been employed in a range of different tumors with success. MTH-68/H has been developed into a highly purified, lyophilized product, containing live, replication competent viral particles, grown to standardized titers. A Phase II clinical trial in humans was completed where the inhalatory mode of administration was used on patients suffering from a variety of advanced malignancies no longer responding to conventional cancer therapies. The study demonstrated relative efficacy, an overall improved quality of life, and a relatively benign side effect profile (Csatary L K, et al., Cancer detection and Prevention, 1993, 17(6):619-627). Success of treatment with MTH-68/H has also been shown in glioblastomas and anaplastic astrocytomas. Glioblastoma multiforme (GBM) is the most common primary brain tumor and by far the most aggressive form of glial tumors. This neoplasm has a poor prognosis, averaging six months to a year without therapy or about one-and-one-half years with conventional therapy. Four cases of advanced GBM and anaplastic astrocytoma were treated with the NDV viral product MTH-68/H after the conventional modalities of cancer therapy had failed. Results included survival rates of twelve and half to six years post-viral treatment for the surviving patients, and six years for one patient who has since died, after having discontinued treatment. Against all odds, these surviving patients returned to good quality of life and to a lifestyle that resemble their pre-morbid daily routines including giving birth to a healthy child and full professional activity. They have been able to enjoy good clinical health. These patients regularly received the MTH-68/H viral therapy for a number of years without interruption as a form of monotherapy once the classical modalities failed. The MRI and CT results have revealed an objective decrease in size of the tumors, in some cases the near total disappearance of the mass.

As reviewed by Zamarin and Palese (Future Microbiol. 2012 March; 7(3): 347-367), when compared to other oncolytic agents in development such as poxvirus, HSV-1, adenovirus, measles, and reovirus vectors, NDV as an oncolytic virus exhibits several advantages over other viruses for use in oncological treatment.

First of all, NDV is an avian pathogen, which avoids the problems of preexisting immunity that can neutralize virus infectivity and of pathogenicity of the virus in humans, which are potential problems with vaccinia, HSV-1, adenovirus, and measles vectors. Serologic studies indicated that ˜96% of the human population is seronegative for NDV, which avoids the problem of pre-existing immunity seen with many human candidate oncolytic viruses. The actual level of seropositivity in today's population may be even lower due to low human exposure to NDV, the outbreaks of which are primarily limited to farm settings. To attest for viral safety, administration of virulent strains to humans has been shown to result in only mild to moderate adverse effects, with mild conjunctivitis, laryngitis, and flu-like symptoms as the only reported infections. Natural human infections with highly virulent (avian) strains have been reported in the literature in farmers and laboratory workers and have been limited to mild symptoms such as conjunctivitis and laryngitis.

Secondly, similarly to other oncolytic viruses, NDV possesses strong immunostimulatory properties that are the basis for oncolytic therapy being considered an immunotherapy. These properties include the induction of type I IFN and chemokines, upregulation of MHC and cell adhesion molecules, and facilitation of adhesion of lymphocytes and antigen-presenting cells (APCs) through expression of viral glycoproteins on the surface of infected cells. These properties have been shown to generate effective anti-tumor immune responses, which may persist long after clearance of the primary viral infection.

Third, the NDV genome has the plasticity to enable the incorporation and stable expression of foreign genes of relatively large size. Since these viruses replicate in the cytoplasm of the cell and not the nucleus, unintended integration of the foreign gene into the host genome is avoided, which could be a safety problem with some DNA oncolytic vectors. Moreover, the absence of homologous RNA recombination ensures that foreign genes expressed from the NDV genome are stable for many serial passages in cell culture and in tumor cells.

Lastly, the ubiquitous nature of the NDV receptor allows for utilization of the virus against a wide variety of tumor types. The specificity of NDV for cancer cells due to their defects in antiviral and apoptotic pathways ensures viral safety and may obviate the need for specific tumor targeting.

Nevertheless, the full view of all clinical data generated to date in different trials of different NDV strains shows that there is much need for improvement in terms of percentage responders and magnitude of therapeutic effect. This need for improvement has spurred efforts to design improved NDV strains, which is the subject of the current application.

Similar to other paramyxoviruses in its family, the 15186, 15192 or 15198 nucleotide(nt)-long negative single-strand RNA genome of NDV encodes six genes including the nucleocapsid protein (NP), phosphoprotein (P), matrix protein (M), fusion protein (F), hemagglutinin-neuraminidase (HN), and RNA-dependent RNA polymerase (L) (Lamb R, Paramyxoviridae: the viruses and their replication. In: Knipe D, editor. Fields Virology. Lippincott Williams & Wilkins; 2007. pp. 1449-1496). The genes are separated by junction sequences that consist of three elements, known as gene start (GS), intergenic (IG), and gene-end (GE) motifs, which regulate mRNA transcription. In the P gene, a unique RNA editing mechanism adds non-templated G residues, resulting in the expression of V and W proteins that are colinear to the P protein in the amino-terminal end. The genomic RNA is bound in a ribonucleotide protein complex (RNP) consisting of NP, P, and L proteins and is surrounded by a lipid envelope containing three virus glycoprotein spikes, HN, M and F. An important factor that influences the extent of virulence of NDV in birds is the cleavage site of the F protein.

Pathogenic classification and virulence of NDV strains in birds generally correlates with their oncolytic properties in human cancer cells. Velogenic (high virulence) strains produce severe respiratory and nervous system signs, spread rapidly through chicken flocks, and can cause up to 90% mortality. Mesogenic (intermediate virulence) strains cause coughing, affect egg production and quality, and can result in up to 10% mortality. Lentogenic (low virulence) strains produce only mild symptoms with little if any mortality. Correspondingly, velogenic strains can efficiently carry out multicycle replication in multiple human cancer cells with effective and efficient cell lysis, lentogenic strains are more attenuated due to lack of activation of the FO protein, and mesogenic strains convey intermediate effects. On the basis of these observations in human cancers, NDV strains have been classified as either lytic or non-lytic, with velogenic and mesogenic viruses being lytic (high and low respectively), and lentogenic viruses in general being non-lytic. Early studies demonstrated that the lytic abilities of lentogenic NDV strains could be enhanced by the introduction of a polybasic cleavage site into their F proteins (Peeters et al, J. Virol. 1999; 73:5001-5009). Other NDV proteins including NP, P, V, HN, and L have also been shown to be implicated in virulence in birds. A deletion (18nt) introduced in the NP gene (Mebatsion T et al., J Virol. 2002 October; 76(20):10138-46) resulted in the deletion of NP residues 443-460. The resulting mutant NDV propagated in embryonated specific-pathogen-free (SPF) chicken eggs to a level fully comparable to that of the parent virus.

In addition, a B-cell epitope of the S2 glycoprotein of murine hepatitis virus (MHV) was inserted in-frame. Recombinant NDV viruses properly expressing the introduced MHV epitope were successfully generated, demonstrating that the NP can be used as the insertion point in the NDV genome to insert foreign or transgenic sequences to be co-expressed with NDV during virus infection.

Several studies showed that type I IFN antagonist activity of NDV V protein is associated with decreased pathogenicity in recombinant viruses possessing attenuating mutations or deletions in the V protein or in viruses expressing no or lower levels of V protein (Huang et al., 2003, J Virol. 77:8676-8685; Mebatsion et al., 2001, J Virol. 75:420-428; Qiu et al., 2016, PLoS ONE 11(2): e0148560. doi: 10.1371/journal.pone.0148560; Elankumaran et al., 2010, J Virol. 84:3835-3844; Jang et al., 2010, Appl Microbiol Biotechnol. 85:1509-1520; Alamares et al., 2010, Virus Res. 147:153-157; Park et al., 2003, J Virol. 77:9522-9532.). It was found to be sufficient to introduce a small (6 nt) deletion in the P-gene to abolish expression of the V-protein (Mebatsion et al., 2001). Recently, two studies showed the role of the polymerase complex in viral pathogenesis, with L protein contributing to pathogenicity of the mesogenic Beaudette C strain, and NP, P, and L proteins contributing to pathogenicity of the velogenic Herts strain (Rout & Samal, 2008, J Virol. 82:7828-7836; Dortmans et al., 2010, J Virol. 84:10113-10120). In addition, several studies noted the contribution of the HN protein to virulence in birds (Römer-Oberclorfer et al., 2006, Avian Dis. 50:259-263; Paldurai et al., 2014, J Virol. 88: 8579-8596.) The HN (hemagglutinin-neuraminidase) protein of NDV is a multifunctional protein with receptor-recognition, hemagglutinin (HA) and receptor-destroying neuraminidase (NA) activities associated with the virus. HN is thought to possess both the receptor recognition of sialic acid at the termini of host glycoconjugates and the neuraminidase activity to hydrolyze sialic acid from progeny virion particles in order to prevent viral self-aggregation. It also recognizes sialic acid-containing receptors on cell surfaces and promotes the fusion activity of the F protein, thereby allowing the virus to penetrate the cell surface. Thus, the HN protein plays a critical role in viral infection of birds.

Reverse genetics (rg) systems to provide recombinant NDV vector virus have been known since 1999 (Peeters et al., 1999, J. Virol. 73:5001-5009), allowing such desired modification and engineering of NDV genomes. Indeed, various reports have shown the successful use of recombinant NDV vectors engineered to express various transgenes (foreign genes incorporated in the genome of the virus) with the goal improving viral oncolytic efficacy (Vigil et al. Cancer Res. 2007, 67:8285-8292; Janke et al., 2007, Gene Ther. 14:1639-1649). However, it should be noted that the replication capability of a recombinant NDV expressing a transgene may be hampered in comparison with its parental NDV strain not expressing the transgene. As discussed above, NDV F protein is responsible for viral fusion with the cell membrane and for viral spread from cell to cell via formation of syncytia. The presence of the multi-basic amino acid cleavage site in the F protein enables protein cleavage and activation by a broad range or proteases and is known to be a determinant of virulence in velogenic NDV strains. In order to increase the oncolytic potency of a highly attenuated lentogenic Hitchner B1 NDV strain, a polybasic cleavage site was introduced into the F protein to generate rNDV/F3aa with mutated F protein. Altomonte et al. (Mol Ther. 2010 18:275-84) reported an oncolytic NDV vector virus with the mutated F-protein rNDV/F(L289A), harboring an L289A mutation within the F protein, which is required for virus entry and cell-cell fusion. This rNDV/F3aa(L289A) virus with mutated F protein showed further enhanced fusion and cytotoxicity of hepatocellular carcinoma (HCC) cells in vitro, as compared with a rNDV/F3aa control virus. In vivo administration of rNDV/F3aa(L289A), via hepatic arterial infusion in immune-competent Buffalo rats bearing multifocal, orthotopic liver tumors resulted in tumor-specific syncytia formation and necrosis, with no evidence of toxicity to the neighboring hepatic parenchyma, which translated to a 20% prolongation of survival time relative to treatment with the original rNDV/F3aa virus.

Due to compelling preclinical data implicating oncolytic NDV as an ideal candidate for cancer therapy, phase I and II clinical trials have been initiated. Several trials have shown that NDV is a safe and potentially therapeutically useful therapeutic agent, with no reports of significant adverse effects in patients beyond conjunctivitis or mild flu-like symptoms (Csatary et al., 1999, Anticancer Res. 19:635-638; Pecora et al., 2002, J. Clin. Oncol. 20:2251-2266; Lorence et al., 2003, Curr. Opin. Mol. Ther. 5, 618-624; Freeman et al., 2006, Mol. Ther. 13:221-228). Although the results of these clinical trials have been encouraging, the extent of clinical responses has not been strong and robust enough to consider bringing one of these initial NDV strains into advanced clinical development. Thus, strategies for improving therapeutic effectiveness while maintaining an acceptable safety profile of oncolytic NDV strains are warranted, and the established system for generating modified NDV vectors through reverse-genetics (rg) has provided a unique opportunity to achieve this aim.

### THE INVENTION

In a first embodiment, the invention provides an oncolytic NDV for use in oncological treatment (this term where used herein is also replaceable with “for use as a medicament, especially in a method of treatment of cancer”; or the use of said oncolytic NDV in the preparation of a pharmaceutical formulation for use in said method of treatment) in humans (or more generically animals, such as mammalian animals), having a mutation in the HN gene, said mutation providing it with an unexpected beneficially increased replication capability in human cancer cells, preferably allowing replication of said oncolytic NDV in a human cancer cell at least 2-fold higher, preferable to ˜2- to 10-fold higher levels, preferably to 5- to 10-fold higher levels than an NDV parent strain not having said mutation. Preferably, said parent strain comprises or is an oncolytic NDV already having an advantageous safety and efficacy profile in humans, such as strain MTH-68/H. In particular, it is used for the treatment of one or more indications selected from the group of brain tumors, like glioblastoma multiforme, astrocytoma bone tumors, like osteosarcoma and/or Ewing's sarcoma, leukemia, lymphoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose & throat (ENT) tumors, tongue tumors, skin tumors, like melanoma, neuroblastoma, mesothelioma, renal cell carcinoma, fibrosarcoma, pheochromocytoma, head and/or neck carcinoma. Preferably the oncolytic NDV may be used for the treatment of adults and/or children.

In one embodiment, the invention encodes an oncolytic NDV having a leucine (L) at position 277 of the HN gene. It is preferred that said mutation in the NA gene encodes the change of a phenylalanine (F) at position 277 of the protein sequence of the parent HN gene to a leucine L in an oncolytic NDV according to the invention. An oncolytic NDV having such a mutation is herein identified as NDV^{F277L} to discern it from a parent NDV having a phenylalanine at position 277 of the HN gene. Also, the invention provides a method to obtain oncolytic NDV having a beneficially increased replication capability in comparison to its NDV parent strain, comprising serially passaging said parent strain virus in a human cancer cell culture, preferably a HeLa cell culture, and harvesting a progeny oncolytic NDV strain having increased replication capability. Preferably the invention provides a method for selecting said NDV^{F277L}. In a preferred embodiment, the invention provides an oncolytic NDV derived from NDV strain MTH-68/H, said oncolytic NDV according to the invention having an unexpected beneficially increased replication capability allowing replication of said oncolytic NDV in a human cancer cell up to ˜10-fold higher levels than oncolytic NDV parent strain MTH-68/H, thereby maintaining the useful oncological safety and efficacy specifications of the parent virus and advantageously supplementing it with the increased replication capability of an oncolytic NDV as provided by the invention herein. Also, the invention provides a method to obtain oncolytic NDV having a beneficially increased replication capability from NDV parent strain MTH-68/H, comprising serially passaging said MTH-68/H parent virus in a human cancer cell culture such as a HeLa cell culture and selecting a progeny oncolytic NDV strain having increased replication capability in comparison to its NDV parent strain. In a much-preferred embodiment, the invention provides oncolytic strain NDV-MutHu, said strain NDV-MutHu derived from NDV strain MTH-68/H, said oncolytic NDV-Muthu having an unexpected beneficially increased replication capability allowing replication of said oncolytic NDV in a human cancer cell culture, preferably a HeLa cell culture to at ˜10-fold higher levels than oncolytic NDV parent strain MTH-68/H. The invention also provides a nucleic acid comprising the nucleic acid sequence of strain NDV-MutHu, having mutations in the nucleic acids encoding viral proteins HN and M (M^{G165W} and HN^{F277L}) in comparison to strain NDV-MTH-68/H. The skilled artisan knows how to use the disclosed sequence to produce synthetic DNAs (e.g. vectors, in particular in rg-NDV vectors), and use the synthetic DNAs to reconstruct and express the viral genome. Thus, the skilled artisan is enabled to produce virus particles by using the information disclosed herein. In a further embodiment, the invention provides a method (herein also called ‘reverse genetics’) for providing cloned full-length NDV-MutHu cDNA, and, in a further embodiment, the invention also provides Infectious virus from said full-length NDV-MutHu cDNA, said infectious virus herein for example designated rgMutHu. Typically, NDV-MutHu and rgMutHu as provided herein have similar unexpected beneficial replication capability in human cancer cells. The invention also provides infectious virus (rgNDV) derived from said full-length NDV-MutHu cDNA, wherein either one or both mutations in viral protein M or HN have been restored, said rgNDV herein designated rgMutHu in which the HN and M mutations have been restored. In one embodiment of the invention, the mutation in the M gene has been restored resulting in infectious copy virus [rgMutHu(M^{W165G})]. In another embodiment of the invention, the mutation in the HN gene has been restored resulting in infectious copy virus [rgMutHu(HN^{L277F})]. In yet another embodiment, of the invention, both the mutation in the M gene as well as that in the HN gene have been restored, resulting in infectious copy virus [rgMutHu(M^{W165G}/HN^{L277F})] as herein provided, which is identical to rgMTH68, which is an infectious copy virus of oncolytic strain MTH-68/H.

Unexpected, and surprisingly, it was then established that only the HN mutation is to be held responsible for the improved growth kinetics of NDV-MutHu. NDV-MutHU infectious copy virus, when compared to its parent strain MTH68, grows to surprisingly higher titers than its parent strain. Even though in HeLa cells MTH68 seems to induce CPE earlier than NDV-MutHu and rgMutHu, unexpectedly NDV-MutHu, rgMutHu and restored rgMutHu(M^{W165G}) each reach ˜5- to 10-fold higher virus titers than parent strain MTH-68/H and its infectious copy virus rgMTH68, together with restored infectious copy virus [rgMutHu(HN^{L277F})] from which the HN-mutation was restored to the HN sequence of the parent strain. The invention thus provides a facile reverse genetics system for NDV wherein genetic modification (including the insertion of therapeutic transgenes or useful deletions) of oncolytic NDV with improved replication capability such as strains NDV-MutHu and restored rgMutHu(M^{W165G}) is possible.

Thus, having unexpectedly established the mutation in the HN gene as necessary and sufficient for beneficially improving replication capability, in a further embodiment, the invention provides a NDV strain mutation in the HN gene that confers an unexpected beneficially increased replication capability, said mutation allowing replication of said NDV in a human cancer cell to higher levels than replication of an otherwise identical NDV not having said beneficial mutation in the HN gene. In a further embodiment, the invention also provides an isolated, synthetic or recombinant nucleic acid encoding rgNDV having or provided with a mutation in the HN gene, said mutation allowing replication of said rgNDV in a human cancer cell to higher levels than replication of an otherwise identical rgNDV not having or not been provided with said mutation in the HN gene. Although the functions of the HN protein in NDV infection in birds have been well studied, its roles in viral replication in human cancer cells and in NDV-mediated oncolysis are not known. The inventors have now unexpectedly established that modifying HN beneficially affects replication of rgNDV in cancer cells. Herewith, the inventors identified a novel mutation in the HN protein of NDV with unexpected and promising utility in oncology. Changing the amino acid at position 277 of the HN protein from F to L increases replication in human cancer cells and increases viral yield of the rgNDV grown in such cells; changing L at position 277 back to F reduces replication and yield of rgNDV.

This finding shows that modifying the HN gene in oncolytic rgNDV strains improves replication, and this allows for several advantageous and novel developments. Firstly, the rgNDV virus provided herein grows to higher than expected titers (yields) in cell cultures, eggs or animals that are used to propagate the virus for pharmaceutical purposes, giving production advantages to such improved rgNDV strains. Secondly, higher titers are very useful biologically to obtain the desired oncolytic effects in vivo in that more virus will be produced for subsequent rounds of infection of cancer cells that were missed in the first round of infection. Thirdly, higher levels (yields) of transgenic proteins will be achieved for providing the desired enhanced oncolytic effects in vivo. Thus, by modifying the HN protein within a transgene-expressing NDV, the invention surprisingly provides a transgene-expressing NDV with increased replication capacity in comparison to that of the patent virus and higher yields of the transgenic construct protein, which is very useful in anti-tumor immunotherapy. The invention also provides a novel method for preparing an rgNDV having improved replication in a cancer cell over a parent NDV, said method comprising providing a nucleic acid construct encoding at least a part of a HN gene with a mutation, incorporating said nucleic acid construct with said mutation into a nucleic acid derived from said parent NDV to produce a nucleic acid encoding an rgNDV, using said nucleic acid encoding a rgNDV to produce infectious rgNDV, comparing the replication characteristics in cancer cells of said infectious rgNDV with the replication characteristics of said parent NDV, and selecting said rgNDV for further use when it shows improved replication characteristics over said parent NDV.

The invention also provides an isolated, synthetic or recombinant nucleic acid encoding a rgNDV obtainable by a method for preparing an rgNDV having improved replication in a cancer cell in comparison to that of a parent NDV, said method comprising providing a nucleic acid construct encoding at least a part of a HN gene with a mutation such as a mutation resulting in a change of the amino acid phenylalanine (F) at amino acid position 277 of the hemagglutinin-neuraminidase, incorporating said nucleic acid construct with said mutation into a nucleic acid derived from said parent NDV to produce a nucleic acid encoding an rgNDV, using said nucleic acid encoding a rgNDV to produce infectious rgNDV, comparing the replication characteristics in cancer cells of said infectious rgNDV with the replication characteristics of said parent NDV, selecting said rgNDV for further use when it shows improved replication characteristics over said parent NDV. In a further embodiment, the invention provides a nucleic acid obtainable with such a method as provided herein, said nucleic acid being an isolated, synthetic or recombinant nucleic acid encoding a rgNDV provided with a mutation in the HN gene resulting in a change of the amino acid phenylalanine (F) at amino acid position 277 of the hemagglutinin-neuraminidase into a leucine (L), said mutation (an F277L mutation) allowing replication of said rgNDV in a human cancer cell to a higher level than replication of an otherwise identical rgNDV having the amino acid phenylalanine (F) at position 277 in the HN gene.

The invention also provides an isolated, synthetic or recombinant nucleic acid encoding a rgNDV having or provided with a mutation in the HN gene, said mutation allowing replication of said rgNDV in a human cancer cell to higher levels than replication of an otherwise identical rgNDV not having or not been provided with said mutation in the HN gene, said nucleic acid additionally having been provided with a transgenic construct. In one embodiment, it is preferred that said transgenic construct provides increased lysis of a cancer cell. In a yet further preferred embodiment, said transgenic construct is selected from the group of nucleic acid constructs encoding a protein that reduces or inhibits IFN expression to improve oncolysis, encoding a protein with cytokine activity to improve oncolysis, encoding a protein with antibody activity to improve oncolysis, encoding a protein with apoptotic activity to improve oncolysis, encoding a protein capable of blocking checkpoint inhibition to improve oncolysis, encoding a green fluorescent protein (GFP) to improve visual detection of infected cells, and encoding a protein capable of enhanced binding (targeting) to a cancer cell to improve oncolysis.

In one much preferred embodiment, the invention provides a nucleic acid obtainable with such a method as provided herein, said nucleic acid being an isolated, synthetic or recombinant nucleic acid encoding a rgNDV provided with a mutation in the HN gene resulting in a change of the amino acid phenylalanine (F) at amino acid position 277 of the hemagglutinin-neuraminidase into a leucine (L), said mutation (an F277L mutation) allowing replication of said rgNDV in a human cancer cell to a higher level than replication of an otherwise identical rgNDV having the amino acid phenylalanine (F) at position 277 in the HN gene, said transgenic construct encodes a protein that reduces or inhibits IFN expression such as an IFN-beta receptor, preferably viral protein B18R from vaccinia virus. B18R is a homologue of the human IFN-β receptor. When B18R is secreted from cells infected with an rNDV expresser strain it acts as a decoy for IFN-β, thereby inhibiting activation by cell signaling of the antiviral host response in naive cells, resulting in increased lytic activity in cancer cells.

In one other much preferred embodiment, the invention provides a nucleic acid obtainable with such a method as provided herein, said nucleic acid being an isolated, synthetic or recombinant nucleic acid encoding a rgNDV provided with a mutation in the HN gene resulting in a change of the amino acid phenylalanine (F) at amino acid position 277 of the hemagglutinin-neuraminidase into a leucine (L), said mutation (an F277L mutation) allowing replication of said rgNDV in a human cancer cell to a higher level than replication of an otherwise identical rgNDV having the amino acid phenylalanine (F) at position 277 in the HN gene, said transgenic construct encodes a protein with apoptotic activity such as viral protein 3 from chicken anemia virus, apoptin. Apoptin (VP3 from chicken anemia virus) has been shown to selectively induce apoptosis when expressed by an rNDV expresser strain in human tumor cells, but not in normal human cells, hence an improved therapeutic index for killing cancer cells and not normal cells.

In one other much preferred embodiment, the invention provides a nucleic acid obtainable with such a method as provided herein, said nucleic acid being an isolated, synthetic or recombinant nucleic acid encoding a rgNDV provided with a mutation in the HN gene resulting in a change of the amino acid phenylalanine (F) at amino acid position 277 of the hemagglutinin-neuraminidase into a leucine (L), said mutation (an F277L mutation) allowing replication of said rgNDV in a human cancer cell to a higher level than replication of an otherwise identical rgNDV having the amino acid phenylalanine (F) at position 277 in the HN gene, said transgenic construct encodes an antibody capable of blocking checkpoint inhibition, such as an antibody directed against checkpoint inhibition protein PD-1 (programmed cell death protein 1, also known as CD279). It is preferred that such an antibody is Nivolumab or a functional analog thereof that works as a checkpoint inhibitor, blocking a signal that prevents activated T cells from attacking the cancer upon expression in rNDV-expresser cells, thus allowing the immune system to clear the cancer better than would the parent NDV. Binding of Nivolumab analogue to PD-1 on T-cells results in the elimination of downregulation of T-cell effector responses by cancer cells.

In a further embodiment, the invention provides a nucleic acid, obtainable with such a method as provided herein, said nucleic acid being an isolated, synthetic or recombinant nucleic acid encoding a rgNDV provided with a mutation in the HN gene wherein said transgenic construct encodes a green fluorescent protein, such as enhanced GFP (EGFP) to allow for tracing in-vitro or in-vivo replication, if desired. In another embodiment, the invention provides a nucleic acid, obtainable with such a method as provided herein, said nucleic acid being an isolated, synthetic or recombinant nucleic acid encoding a rgNDV provided with a mutation in the HN gene wherein said transgenic construct encodes a protein capable of providing specific binding (targeting) of rgNDV to a cancer cell, such as a modified HN protein that is fused to a single-chain antibody against a tumor-associated cell surface protein, a modified HN protein that is fused to a ligand that specifically binds to a tumor-associated cell-surface protein, or a bi-specific protein consisting of a single-chain antibody against the rgNDV HN protein fused to a single-chain antibody or ligand that specifically binds to a tumor-associated cell-surface protein thereby increasing the therapeutic window for treatment of cancer with this recombinant expresser strain.

In another embodiment, the invention provides a nucleic acid, obtainable with such a method as provided herein, said nucleic acid being an isolated, synthetic or recombinant nucleic acid encoding a rgNDV provided with a mutation in the HN gene, said nucleic acid additionally having been provided with a mutation in the F gene, said mutation capable of improving oncolytic potential of said rgNDV. In a further preferred embodiment, the invention provides a nucleic acid, obtainable with such a method as provided herein, said nucleic acid being an isolated, synthetic or recombinant nucleic acid encoding a rgNDV provided with a mutation in the HN gene, said nucleic acid additionally having been provided with a mutation in the F gene, said mutation capable of improving oncolytic potential of said rgNDV, wherein said mutation in the F gene comprises or is an L289A mutation.

In yet another embodiment, the invention provides a nucleic acid, obtainable with such a method as provided herein, said nucleic acid being an isolated, synthetic or recombinant nucleic acid encoding a rgNDV provided with a mutation in the HN gene, said nucleic acid additionally having been provided with a mutation in the RNA-editing sequence of the P gene that abolishes or decreases the expression of the V protein.

In yet another embodiment, the invention provides a nucleic acid, obtainable with such a method as provided herein, said nucleic acid being an isolated, synthetic or recombinant nucleic acid encoding a rgNDV provided with a mutation in the HN gene, said nucleic acid additionally having been provided with a mutation in the RNA-editing sequence of the P gene that abolishes or decreases the expression of the V protein.

The invention also provides an rgNDV comprising a nucleic acid according to the invention. Also, the invention provides a method for preparing a rgNDV having improved replication in a cancer cell over a parent NDV, said method comprising the steps: providing a nucleic acid construct encoding a HN gene with a mutation, incorporating said nucleic acid construct with said mutation in a nucleic acid encoding a rgNDV, using said nucleic acid encoding a rgNDV to produce infectious rgNDV, comparing the replication characteristics in cancer cells of said rgNDV with the replication characteristics of said parent NDV, selecting said rgNDV for further use when it shows improved replication characteristics over said parent NDV.

The invention also provides an rgNDV comprising a nucleic acid according to the invention additionally having been provided with a transgenic construct. Also, the invention provides a method for preparing a rgNDV having improved replication in a cancer cell over a parent NDV, said method comprising the steps: providing a nucleic acid construct encoding a HN gene with a mutation, incorporating said nucleic acid constrict with said mutation in a nucleic acid encoding a rgNDV, using said nucleic acid encoding a rgNDV to produce infectious rgNDV, comparing the replication characteristics in cancer cells of said rgNDV with the replication characteristics of said parent NDV, selecting said rgNDV for further use when it shows improved replication characteristics over said parent NDV, and further comprising providing said nucleic acid encoding a rgNDV of step b with a transgenic construct, preferably wherein said transgenic construct provides improved lysis of a cancer cell. In one embodiment, it is preferred that said transgenic construct is selected from the group of nucleic acid constructs encoding a protein that reduces or inhibits IFN expression, encoding a protein with cytokine activity, encoding a protein with antibody activity, encoding a protein with apoptotic activity, encoding a protein capable of blocking checkpoint inhibition, encoding GFP, and encoding a protein capable of enhanced binding (targeting) to a cancer cell. In another embodiment, said nucleic acid encoding a rgNDV of step b is also provided with a mutation in the F gene, said mutation capable of improving oncolytic potential of said rgNDV, in particular wherein said mutation in the F gene comprises or is an L289A mutation. In a further embodiment, said nucleic acid encoding a rgNDV of step b is also provided with a mutation in the RNA-editing sequence of the P gene that abolishes or decreases the expression of the V protein.

The invention also provides a method for preparing a rgNDV having improved replication in a cancer cell over a parent NDV, said method comprising the steps: providing a nucleic acid construct encoding a HN gene with a mutation, incorporating said nucleic acid constrict with said mutation in a nucleic acid encoding a rgNDV or transgene-expressing NDV with increased replication capacity as provided herein, using said nucleic acid encoding a rgNDV to produce infectious rgNDV, comparing the replication characteristics in cancer cells of said rgNDV with the replication characteristics of said parent NDV, selecting said rgNDV for further use when it shows improved replication characteristics over said parent NDV, and further comprising providing said nucleic acid encoding a rgNDV of step b with a transgenic construct, preferably wherein said transgenic construct provides improved lysis of a cancer cell, the method further comprising providing said nucleic acid encoding a rgNDV of step b with a decreased or abolished expression of the V protein. The invention also provides an rgNDV having improved replication in a cancer cell obtainable by a method for preparing a rgNDV having improved replication in a cancer cell over a parent NDV as provided, said method comprising the steps: providing a nucleic acid construct encoding a HN gene with a mutation, incorporating said nucleic acid constrict with said mutation in a nucleic acid encoding a rgNDV, using said nucleic acid encoding a rgNDV to produce infectious rgNDV, comparing the replication characteristics in cancer cells of said rgNDV with the replication characteristics of said parent NDV, selecting said rgNDV for further use when it shows improved replication characteristics over said parent NDV, and further comprising providing said nucleic acid encoding a rgNDV of step b with a transgenic construct, preferably wherein said transgenic construct provides improved lysis of a cancer cell. The invention also provides a pharmaceutical formulation comprising an rgNDV having improved replication in a cancer cell as provided herein without or preferably with at least one pharmaceutically acceptable carrier material.

For the indications mentioned herein, the appropriate dosage will, of course, vary depending upon, for example, the particular molecule of the invention to be employed, the mode of administration and the nature and severity of the condition being treated. In general, the dosage, in one embodiment of the invention, can preferably be in the range of 10⁷ to 10⁹ pfu per dose.

In the above cases of the new NDV strains according to the current invention, improved replication capacity in cancer cells would be associated with increased cancer cell lysis and increased anti-cancer activity relative to activity in normal cells would be associated with an increased therapeutic window or safety margin, as is commonly determined for cancer therapeutics.

In another embodiment, the invention provides a method for producing a pharmaceutical formulation, the method comprising: propagating an rgNDV having improved replication in a cancer cell as provided herein in at least one cell or embryonated egg that is susceptible to a NDV infection; collecting the progeny virus, wherein the virus is grown to sufficient quantities and under sufficient conditions that the virus is free from exogenous contamination, such that the progeny virus is suitable for formulation into a pharmaceutical formulation; and preferably manufacturing a pharmaceutical formulation comprising said progeny virus in the absence of or after addition of at least one pharmaceutically acceptable carrier material.

Pharmaceutical formulations (=pharmaceutical compositions) of the invention may be manufactured in conventional manner. E.g. a composition according to the invention comprising the virus of the invention is provided in lyophilized form and can be complemented with an aqueous carrier for injection when used for administration. For immediate administration it is directly dissolved in a suitable aqueous carrier, for example sterile water for injection or sterile buffered physiological saline.

The invention in a further embodiment also provides a cell or cell line or an embryonated egg comprising a rgNDV having improved replication in a cancer cell as provided herein and in another embodiment a method for treating (=method of treatment of) cancer, comprising administering to a subject in need thereof a rgNDV having improved replication in a cancer cell as provided herein.

In describing nucleic acid formulation, structure and function herein, reference is made to any of a group of polymeric nucleotides such as DNA or RNA. Nucleic acid is composed of either one or two chains of repeating units called nucleotides, which consist of a nitrogen base (a purine or pyrimidine) attached to a sugar phosphate. In the present specification, nucleotide residues are identified by using the following abbreviations. Adenine residue: A; guanine residue: G; thymine residue: T; cytosine residue: C; uracil residue: U. Also, unless explicitly otherwise indicated, the nucleotide sequences of nucleic acid are identified from 5′-terminal to 3′-terminal (left to right terminal), the 5′-terminal being identified as a first residue.

In describing protein or peptide formulation, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal (left terminal to right terminal), the N-terminal being identified as a first residue. Amino acids are designated by their 3-letter abbreviation, 1-latter abbreviation, or full name, as follows. Ala:A:alanine; Asp:D:aspartic acid; Glu:E:glutamic acid; Phe:F:phenylalanine; Gly:G:glycine; His:H:histidine; Ile:I:isoleucine; Lys:K:lysine; Leu:L:leucine; Met:M:methionine; Asn:N:asparagine; Pro:P:proline; Gln:Q:glutamine; Arg:R:arginine; Ser:S:serine; Thr:T:threonine; Val:V:valine; Trp:W:tryptophan; Tyr:Y:tyrosine; Cys:C:cysteine.

In another aspect, presented herein are methods for propagating the NDVs described herein (e.g., rgNDVs) in any cell, subject, tissue or organ susceptible to a NDV infection. In one embodiment, the NDVs described herein may be propagated in a cell line. In another embodiment, the NDVs described herein may be propagated in cancer cells. In another embodiment, the NDVs described herein may be propagated in an embryonated egg. In certain embodiments, presented herein are isolated cells, tissues or organs infected with an NDV described herein. In specific embodiments, presented herein are isolated cancer cells infected with an NDV described herein. In certain embodiments, presented herein are cell lines infected with an NDV described herein. In other embodiments, presented herein are embryonated eggs infected with an NDV described herein.

In another aspect, presented herein are formulations comprising an NDV described herein. In a specific embodiment, presented herein are pharmaceutical formulations comprising an NDV described herein and a pharmaceutically acceptable carrier for injection and/or stabilization of the rgNDV and/or improving its delivery to cancer cells. In particular it may be intended for being used for the treatment of one or more indications selected from the group of brain tumors, like glioblastoma and/or astrocytoma, leukemia, lymphoma, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose throat tumors, tongue tumors, skin tumors, like melanoma, neuroblastoma, mesothelioma, renal cell carcinoma, fibrosarcoma, pheochromocytoma, head and/or neck carcinoma in adults and/or children. In another embodiment, presented herein are pharmaceutical formulations comprising cancer cells infected with an NDV described herein (e.g., a rgNDV or transgene-expressing NDV with increased replication capacity as described herein), and a pharmaceutically acceptable carrier. In specific embodiments, the cancer cells have been treated with gamma radiation prior to incorporation into the pharmaceutical formulation. In specific embodiments, the cancer cells have been treated with gamma radiation before infection with the NDV (e.g., rgNDV). In other specific embodiments, the cancer cells have been treated with gamma radiation after infection with the NDV (e.g., rgNDV). In another embodiment, presented herein are pharmaceutical formulations comprising protein concentrate from lysed NDV-infected cancer cells (e.g., rgNDV infected cancer cells), and a pharmaceutically acceptable carrier.

In another aspect, presented herein are methods for producing a pharmaceutical formulation comprising an NDV described herein (e.g., a rgNDV or transgene-expressing NDV with increased replication capacity as described herein). In one embodiment, a method for producing a pharmaceutical formulation comprises or consists of: (a) propagating an NDV described herein (e.g., a rgNDV or transgene-expressing NDV with increased replication capacity as described herein) in a cell line that is susceptible to an NDV infection; and (b) collecting the progeny virus and processing the collected virus-containing material to enrich virus and/or eliminate host cell DNA, wherein the virus is grown to sufficient quantities and under sufficient conditions that the virus is free from contamination, such that the progeny virus is suitable for formulation into a pharmaceutical formulation. In another embodiment, a method for producing a pharmaceutical formulation comprises or consists of: (a) propagating an NDV described herein in an embryonated egg; and (b) collecting the progeny virus, wherein the virus is grown to sufficient quantities and under sufficient conditions that the virus is free from contamination, such that the progeny virus is suitable for formulation into a pharmaceutical formulation.

In another aspect, presented herein are methods for treating cancer, especially selected from the specific cancers as described and/or mentioned in the claims herein, utilizing a rgNDV described herein or a pharmaceutical formulation comprising such a rgNDV, especially in a therapeutically effective amount to a patient in need thereof. In a specific embodiment, a method for treating cancer comprises or consists of infecting a cancer cell in a subject with a rgNDV described herein or a formulation thereof. In another embodiment, a method for treating cancer comprises or consists of administering to a subject in need thereof a rgNDV described herein or a pharmaceutical formulation thereof by intravenous, intra-arterial, intratumoral, intramuscular, intradermal, subcutaneous, or any other medically relevant route of administration. In specific embodiments, an effective amount of a rgNDV described herein or a formulation comprising a therapeutically (including preventive) effective amount of a rgNDV described herein is administered to a subject to treat cancer.

In another embodiment, a method for treating cancer, especially selected from the specific cancers as described and/or mentioned in the claims herein, comprises or consists of administering to a subject in need thereof cancer cells infected with a rgNDV or a or transgene-expressing NDV with increased replication capacity as described herein, especially in a therapeutically effective amount, or pharmaceutical formulation thereof by intravenous, intra-arterial, intratumoral, intramuscular, intradermal, subcutaneous, or any other medically relevant route of administration. In specific embodiments, the cancer cells have been treated with gamma radiation prior to administration to the subject or incorporation into the pharmaceutical formulation. In another embodiment, a method for treating cancer comprises or consists of administering to a subject in need thereof protein concentrates or plasma membrane fragments from cancer cells infected with a rgNDV or a pharmaceutical formulation thereof.

In another aspect, presented herein are methods for treating cancer, especially selected from the specific cancers as described and/or mentioned in the claims herein, utilizing an NDV described herein or a pharmaceutical formulation comprising such NDV in combination with one or more other therapies. In one embodiment, presented herein are methods for treating cancer, especially selected from the specific cancers as described and/or mentioned in the claims herein, comprising administering to a subject an rgNDV or a transgene-expressing NDV with increased replication capacity as described herein and one or more other therapies (this term wherever used means therapies and/or therapeutics). In another embodiment, presented herein are methods for treating cancer comprising administering to a subject (especially therapeutically) an effective amount of an rgNDV described herein or a pharmaceutical formulation comprising an effective amount of an rgNDV described herein, and one or more other therapies. The rgNDV and one or more other therapies can be administered concurrently or sequentially to the subject (meaning they are jointly therapeutically active). In certain embodiments, the rgNDV and one or more other therapies are administered in the same (“fixed”) pharmaceutical formulation. In other embodiments, the rgNDV and one or more other therapies are administered in different formulations. The rgNDV or transgene-expressing NDV with increased replication capacity as provided herein and one or more other therapies can be administered by the same or different routes of administration to the subject.

### FIGURE LEGENDS

FIG. 1. NDV reverse genetics

Schematic presentation of the NDV reverse genetics system. The upper part shows the composition of the Full-length cDNA plasmid which contains the full-length NDV cDNA (encoding the NP, P, M, F, HN, and L proteins) cloned behind the bacteriophage T7 RNA Polymerase promoter (T7P; yellow triangle) and followed by a ribozyme sequence (Rz) and T7 transcription termination signal (T7T). A suitable host cell (shaded round-cornered box) is infected with a recombinant Fowlpox virus that expresses T7 DNA-dependent RNA Polymerase (Fowlpox-T7) and subsequently co-transfected with the full-length cDNA plasmid and three helper plasmids containing the genes encoding the NDV NP, P and L proteins, respectively. Transcription of the full-length cDNA results in the generation of the NDV antigenome RNA which is encapsidated by NP protein then transcribed and replicated by the RNA-dependent RNA Polymerase complex consisting of the L and P proteins, ultimately leading to the generation of infectious NDV.

FIG. 2. Assembly of full-length NDV cDNA

Schematic presentation showing the positions and sizes of the subgenomic cDNA fragments that were used to assemble the complete NDV cDNA in the transcription vector pOLTV5 (Peeters et al., 1999, J. Virol. 73:5001-5009). The dots indicate the positions of the G165W mutations in the M gene and the F277L mutation in the HN gene, respectively.

FIG. 3. Growth kinetics in HeLa cells

HeLa cells were infected at a multiplicity of infection (MOI) of 0.01 with the indicated viruses. At 0 h, 8 h, 24 h and 48 h after infection, the amount of infectious virus in the supernatant was determined by end-point titration on QM5 cells. MTH68: NDV strain MTH-68/H; MutHu: NDV strain MutHu; rgMTH68: NDV strain MTH-68/H derived by reverse genetics from cloned full-length cDNA; rgMutHu; NDV strain MutHu derived by reverse genetics from cloned full-length cDNA; rgMutHu(HNL277F); rgMutHu in which the amino acid mutation at position 277 in the HN gene was converted from L back to F; rgMutHu(MW165G): rgMutHu in which the amino acid mutation at position 165 in the M gene was converted from W back to G.

FIG. 4. Growth kinetics in HeLa cells

HeLa cells were infected at a MOI of 0.01 (left panel) or 1.0 (right panel) with the indicated viruses. At 0 h, 8 h, 24 h and 48 h after infection, the amount of infectious virus in the supernatant was determined by end-point titration on QM5 cells. MTH68: NDV strain MTH-68/H; MutHu: NDV strain MutHu; rgMutHu; NDV strain MutHu derived by reverse genetics from cloned full-length cDNA.

FIG. 5. Onset of cytopathogenic effect in NDV-infected HeLa cells

HeLa cells were infected at a MOI of 0.01 (left) or 1.0 (right) with the indicated viruses. At 24 h and 48 h after infection photographs were taken using a light microscope in order to examine the extent of the cytopathogenic effect caused by the different viruses. MTH68: NDV strain MTH-68/H; MutHu: NDV strain MutHu rgMutHu, NDV strain MutHu derived by reverse genetics from cloned full-length cDNA.

FIG. 6. Schematic presentation of the genomes of the rgMutHu strains.

The figure shows the position of the genes encoding Apoptin, B18R and Nivolumab which were inserted as extra transcription units into the NDV rgMutHu genome between the P and M genes.

FIG. 7. Expression of Apoptin by rgMutHu-Apoptin

Expression of Apoptin was verified by means of immunological staining using monoclonal antibody (MAb) CVI-CAV-111.3 against VP3 of chicken anemia virus (Danen-Van Oorschot, 1997, Proc Natl Acad Sci USA. 94: 5843-5847). Briefly, rgMutHu-Apoptin infected cell monolayers were fixed and incubated with horse-radish peroxidase (HRPO)-conjugated MAb CVI-CAV-111.3 for 1 h, and after washing the monolayers, binding of the MAb to Apoptin was detected by HPRO-assay using 3-amino-9-ethylcarbazole (AEC) as a substrate. (A) Non-infected QM5 cells, (B) QM5 cells infected with rgMutHu-Apoptin.

FIG. 8. Expression of Nivolumab by rgNDV-Nivolumab

Undiluted and diluted samples from the supernatant harvested after 48 h from rgMutHu-Nivolumab or rgMutHu infected HeLa cells were used to quantify the amount of Nivolumab using a commercial human IgG4 ELISA kit (ThermoFischer Scientific, catalog number: 88-50590-22). The values in the table represent the OD values and the corresponding standard curve is shown in the right panel. The expression level of Nivolumab amounted to approx. 2000 ng/m L.

FIG. 9. Real-time cell analysis (RTCA) profiles from HeLa cells infected with different NDV strains at an MOI of 0.01

The growth of uninfected or NDV-infected HeLa cells was examined by using a RTCA device (iCELLigence™; ACEA Biosciences Inc.) which measures differences in cellular impedance. The functional unit of a cellular impedance assay is a set of gold microelectrodes fused to the bottom surface of a cell culture plate well. When submersed in a current-conductive solution (such as buffer or standard tissue culture medium), the application of an electric potential across these electrodes causes electrons to exit the negative terminal, pass through bulk solution, and then deposit onto the positive terminal to complete the circuit. Because this phenomenon is dependent upon the electrodes interacting with bulk solution, the presence of adherent cells at the electrode-solution interface impedes electron flow. The magnitude of this impedance is dependent on the number of cells, the size and shape of the cells, and the cell-substrate attachment quality. Importantly, neither the gold microelectrode surfaces nor the applied electric potential has an effect on cell health or behavior (https://www.aceabio.com/products/icelligence/).

Cells were seeded in the wells of the analysis plate and 24 hr later, cells were infected with the indicated viruses at a MOI of 0.01 and further analyzed for 72 h. MTH68: NDV strain MTH-68/H; MutHu: NDV strain MutHu; rgMTH68: NDV strain MTH-68/H derived by reverse genetics from cloned full-length cDNA; rgMutHu; NDV strain MutHu derived by reverse genetics from cloned full-length cDNA; Apoptin: rgMutHu containing the Apoptin gene; Nivolumab; rgMutHu containing the Nivolumab gene; B18R: rgMutHu containing the B18R gene; HeLa: uninfected HeLa cells.

FIG. 10. RTCA profiles from HeLa cells infected with different NDV strains at an MOI of 0.01

The growth of uninfected or NDV-infected HeLa cells was examined by using a RTCA device. Cells were seeded in the wells of the analysis plate and 24 hr later the cells were infected with the indicated viruses at a MOI of 0.1 and further analyzed for 72 h. MTH68: NDV strain MTH-68/H; MutHu: NDV strain MutHu; rgMTH68: NDV strain MTH-68/H derived by reverse genetics from cloned full-length cDNA; rgMutHu; NDV strain MutHu derived by reverse genetics from cloned full-length cDNA; Apoptin: rgMutHu containing the Apoptin gene; Nivolumab; rgMutHu containing the Nivolumab gene; B18R: rgMutHu containing the B18R gene; HeLa: uninfected HeLa cells.

FIG. 11. Cell viability at different time points after infection (MOI=0.1)

The percentage of viable cells was examined at different time points after infection of HeLa cells with the indicated viruses at a MOI of 0.1. Cell viability was determined by means of the trypan-exclusion assay using a Countess II FL Automated Cell Counter (ThermoFischer).

FIG. 12. Virus titers at different time points after infection (MOI=0.1)

HeLa cells were infected with the indicated viruses at a MOI of 0.1 and infectious virus titers in the supernatant were determined at 0 h, 24 h, 48 h and 72 h after infection by end-point titration on QM5 cells. MTH68: NDV strain MTH-68/H; MutHu: NDV strain MutHu; rgMTH68: NDV strain MTH-68/H derived by reverse genetics from cloned full-length cDNA; rgMutHu; NDV strain MutHu derived by reverse genetics from cloned full-length cDNA; rgMutHu-Apoptin: rgMutHu containing the Apoptin gene; rgMutHu-Nivolumab; rgMutHu containing the Nivolumab gene; rgMutHu-B18R: rgMutHu containing the B18R gene.

FIG. 13. Appendix 2: alignment of 5′-terminal sequences

### DETAILED DESCRIPTION

### Example 1. Nucleotide Sequence Analysis of Mutant NDV-MutHu

Newcastle Disease Virus (NDV) is a non-segmented, negative-sense, single-stranded RNA virus belonging to the paramyxovirus family. The natural hosts of NDV are avian species, in particular water- and shorebirds. Some strains of NDV can cause severe and lethal disease in terrestrial poultry such as chickens and turkeys. Infection of humans is rare and is either asymptomatic or limited to a mild and transient conjunctivitis. NDV strains can be categorized into three different groups (pathotypes) based on their pathogenicity for day-old chickens: lentogenic (avirulent), mesogenic (intermediate virulent) and velogenic (virulent). These differences in pathogenicity can largely be explained by differences in the cleavage site in the fusion (F) protein. Lentogenic viruses have a monobasic cleavage site, which can only be cleaved by extra-cellular trypsin-like proteases found in the respiratory and digestive tracts of birds. Mesogenic and velogenic strains have a polybasic cleavage site, which can be cleaved by intra-cellular furin-like proteases, found more abundantly in most cell and organs, explaining why virulent strains can replicate systemically in birds. Several naturally occurring NDV strains have been shown to possess oncolytic anti-tumor properties. In humans, NDV selectively replicates in tumor cells and kills these cells while sparing normal cells. The ratio of killing of cancer cells to normal cells is the therapeutic index for a drug, and the higher it is, the better the efficacy of the drug while minimizing safety-related side effects. The selective replication of NDV in tumor cells is based on several mechanisms, including defects in activation of anti-viral signaling pathways, defects in type-I IFN-signaling pathways, defects in apoptotic pathways, and activation of Ras signaling and expression of Rac1 protein (for recent reviews, see: Schirrmacher, 2015, Expert Opin. Biol. Ther. 15:17 57-71; Zamarin & Palese, 2012, Future Microbiol. 7:347-367).

We identified a spontaneous mutant of an oncolytic NDV strain MTH-68/H (Csatary et al., 1999, Anticancer Res. 19:635-638.; further called MTH68). The replication capacity of the mutant strain (designated NDV-MutHu) in a variety of human neoplastic cell lines, as well as autologous primary tumors, is greatly enhanced as compared to the original MTH68 strain. We analyzed its nucleotide sequence and found that, compared to MTH68, NDV-MutHu has two nucleotide mutations, one leading to an amino acid substitution in the M protein (G165W) and the other in the HN protein (F277L).

### Example 2. A Reverse Genetics System that Allows Genetic Modification of NDV-MutHu

2.1 Reverse Genetics

In order to be able to genetically modify the genome of an RNA virus such as NDV, a manipulatable genetic system must be developed that uses a copy of the full viral RNA (vRNA) genome in the form of DNA. This full-length cDNA is amenable to genetic modification by using recombinant DNA techniques. The authentic or modified cDNA can be converted back into vRNA in cells, which in the presence of the viral replication proteins results in the production of a new modified infectious virus. Such ‘reverse genetics systems’ have been developed in the last few decades for different classes of RNA viruses. This system enables the rapid and facile introduction of mutations and deletions and the insertion of a transgene transcriptional unit, thereby enabling the changing of the biological properties of the virus.

Reverse genetics systems for several NDV strains, including lentogenic as well as velogenic strains, were developed by the Central Veterinary Institute (CVI), part of Wageningen University and Research, currently Wageningen Bioveterinary Research (WBVR) under the supervision of Dr. Ben Peeters (Peeters et al., 1999, J. Virol. 73:5001-9; de Leeuw et al., 2005, J. Gen. Virol. 86:1759-69; Dortmans et al., 2009, J. Gen. Virol. 90:2746-50). In order to generate a reverse genetics system for NDV-MutHu, a similar approach was used. Details of the procedure can be found in the above cited papers and in the paragraphs below. Briefly, the system consists of 4 components, i.e., a transcription plasmid containing the full-length (either authentic or genetically modified) cDNA of the virus, which is used to generate the vRNA, and 3 expression plasmids (‘helper plasmids’) containing the NP, P and L genes of NDV respectively, which are used to generate the vRNA-replication complex (consisting of NP, P and L proteins). Transcription of the cDNA (i.e. conversion of the cDNA into vRNA) and expression of the NP, P and L genes by the helper plasmids is driven by a T7 promoter. The corresponding T7 DNA-dependent RNA polymerase (T7-RNAPol) is provided by a helper-virus (Fowlpox-T7).

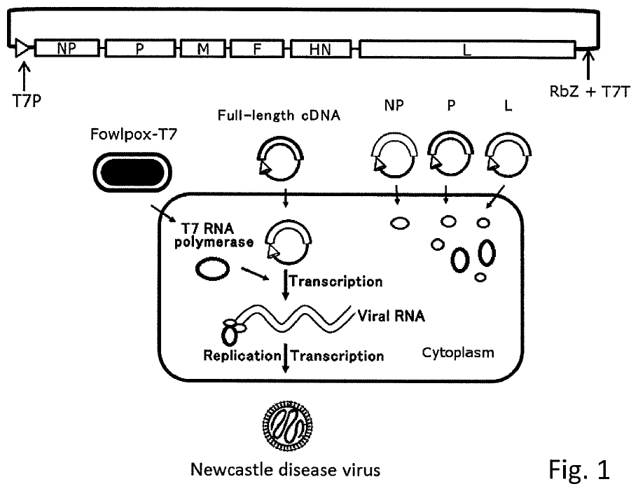

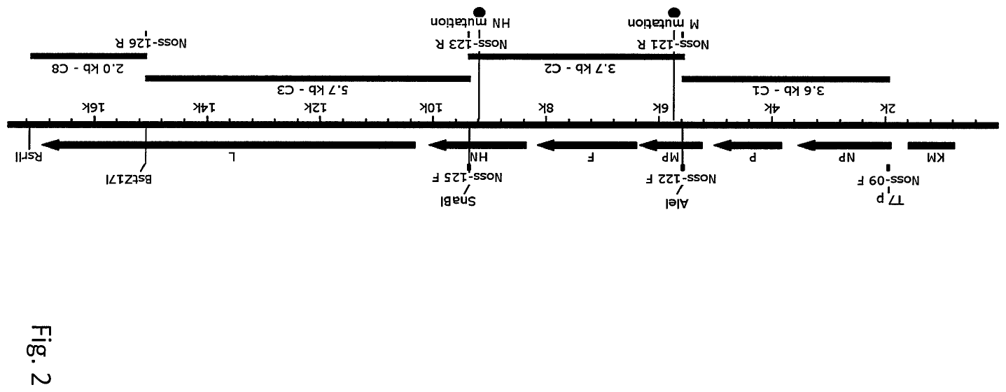

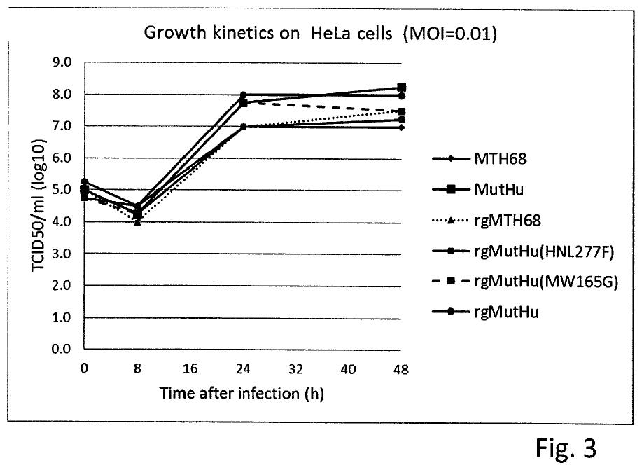

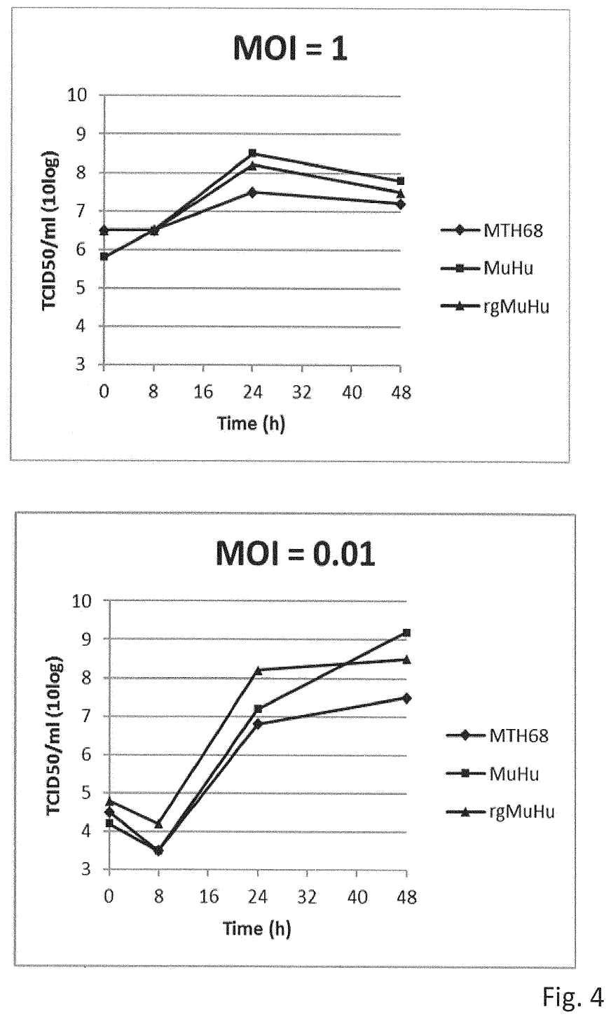

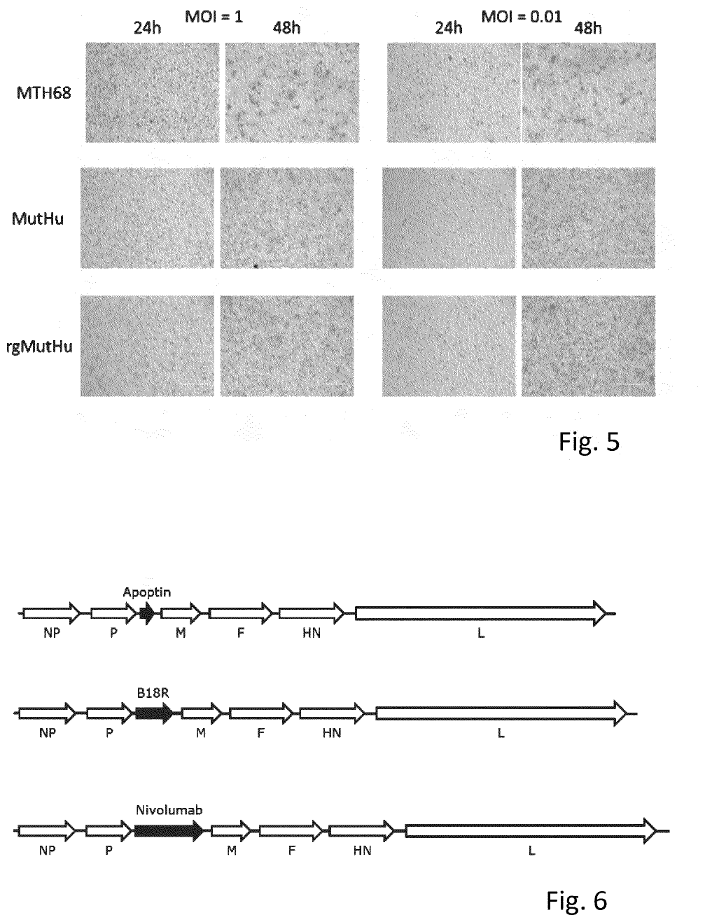

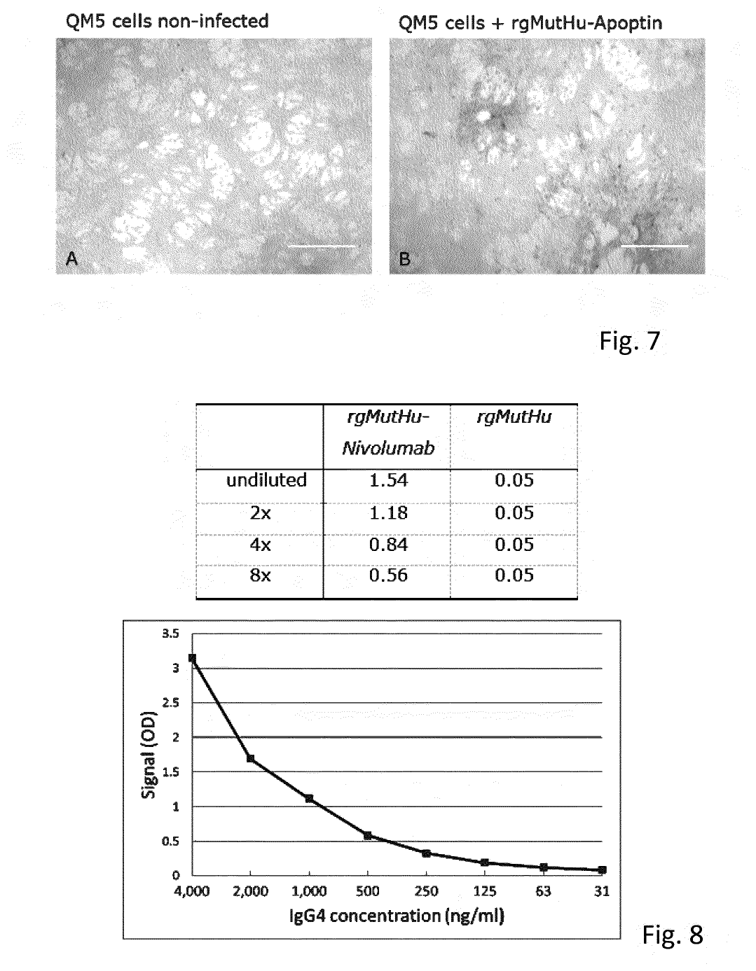

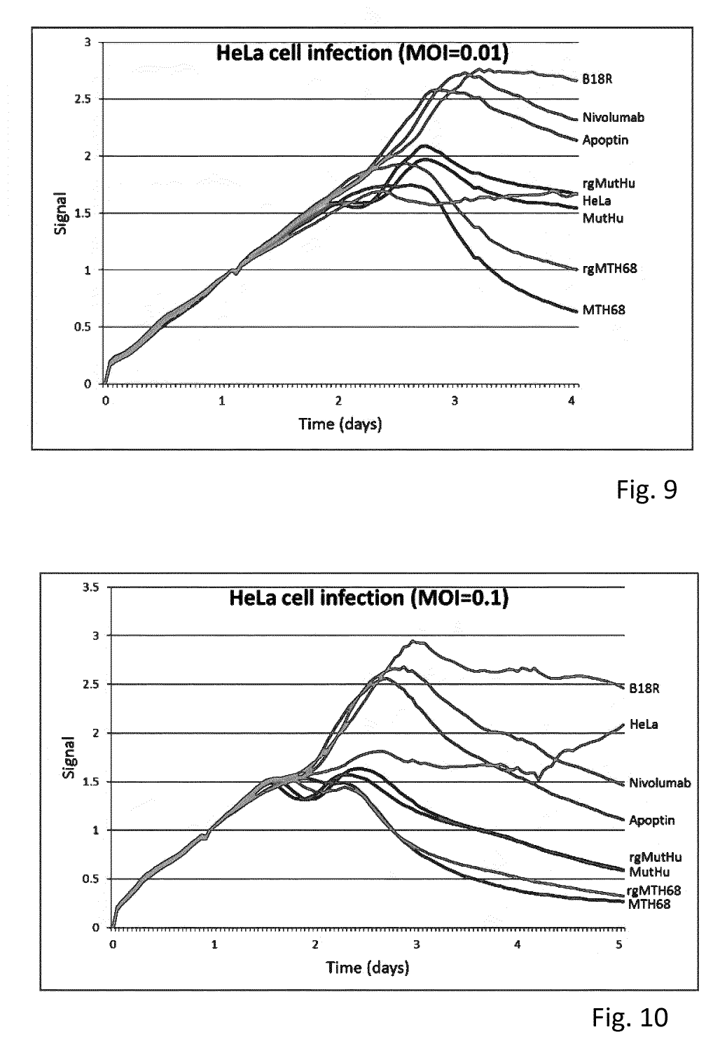

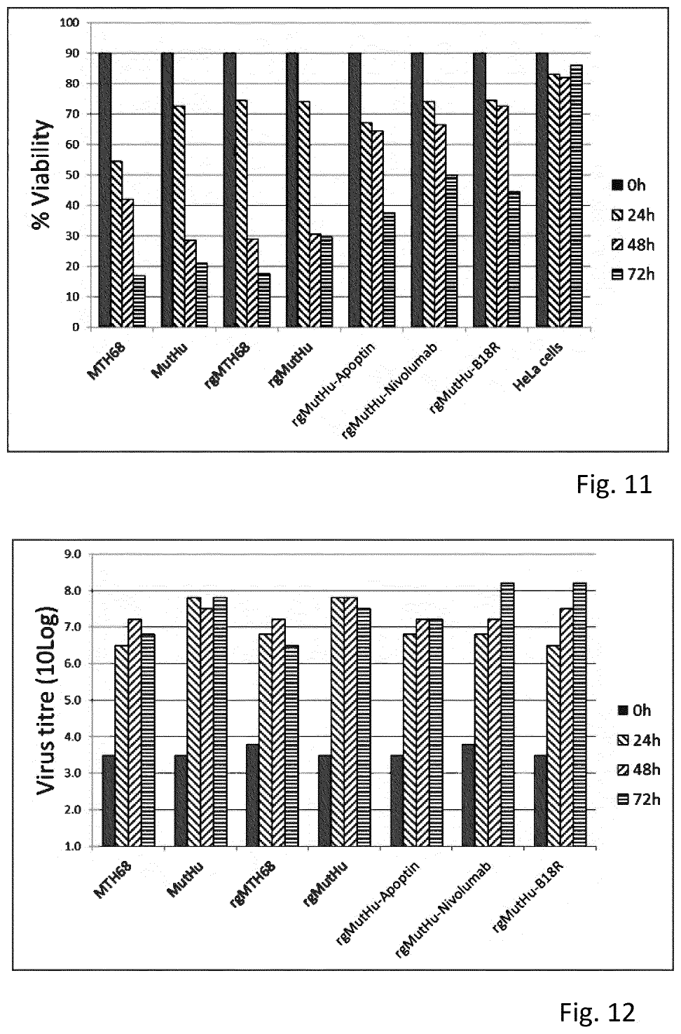

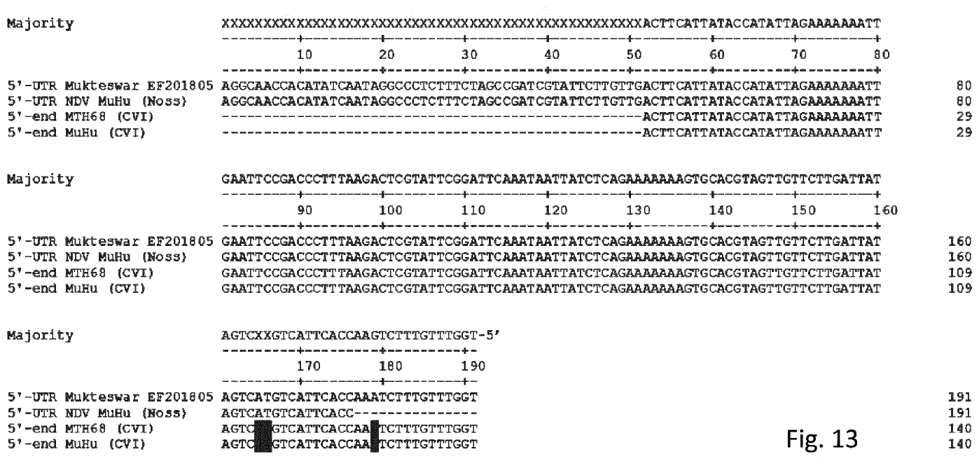

In order to rescue virus, the 4 plasmids are co-transfected into Fowlpox-T7 infected cells (FIG. 1). Three to five days after transfection, the supernatant is inoculated into specific-pathogen-free embryonated chicken eggs (ECE) and incubated for 3 days. Infectious virus that is produced by transfected cells will replicate in the ECE and progeny virus can be harvested from the allantois fluid.

In order to develop a reverse genetics system for NDV-MutHu the following steps were followed:
Generation of sub-genomic NDV-MutHu cDNA's by RT-PCR
Assembly of full-length cDNA in a transcription vector
Cloning of each of the NP, P and L genes into an expression vector
Verify nucleotide sequence of full-length cDNA and helper-plasmids
Repair nucleotide differences resulting from the cloning procedure, if necessary
Rescue of infectious virus from cDNA using co-transfection (FIG. 1)

2.2 Construction of Full-Length NDV-MutHu cDNA and Helper Plasmids

NDV-MutHu (passage 28 HeLa cells) was used for the isolation of vRNA using standard procedures. The vRNA was used to generate first-strand cDNA by means of Reverse Transcriptase followed by PCR to generate 4 sub-genomic cDNA fragments (designated C1, C2, C3 and C8). The full-length cDNA of NDV-MutHu was assembled from these fragments and cloned in the transcription vector pOLTV5 (Peeters et al., 1999, J. Virol. 73:5001-5009) by a combination of In-Fusion® cloning and classical cloning using restriction enzymes. An overview of the procedure is shown in FIG. 2, and further details can be found in Appendix 1 of this reference. The resulting plasmid was designated pFL-NDV_MutHu. The NP, P and L-genes of NDV-MutHu were obtained by RT-PCR (Appendix 1) and cloned in the expression plasmid pCVI which was derived by deletion of a Clal restriction fragment from pCI-neo (Promega). The resulting helper plasmids were designated pCVI-NP^{MutHu}, pCVI-P^{MutHu} and pCVI-L^{MutHu}.

2.3 Nucleotide Sequence Analysis

Nucleotide sequence analysis was used to verify that the sequence of pFL-NDV MutHu was correct. A few nucleotides which differed from the Reference sequence were repaired (Table 1). These mutations may represent a minority species in the original virus stock or may be the result of the technical approach such as misincorporation of nucleotides during reverse-transcription and/or PCR-amplification. Two silent mutations (i.e., not leading to an amino acid change) were left unchanged (nt 1318 and 2339; see Table 1).

We noted a deletion of 1 nt in the Reference sequence compared to the rgMutHu sequence at position 11749. Since this deletion would result in a frameshift in the L-gene (and as a consequence a prematurely terminated L protein), we verified the nucleotide sequence of this region in the NDV-MutHu virus stock. The deletion was not present in the virus sequence and thus probably represents a sequencing error in the Reference sequence. A difference was also noted at position 13529 in the L gene sequence (T in Reference sequence; C in rgMutHu). This region was also verified several times using cDNA from 3 independent RT reactions. Each time the nucleotide at this position proved to be a C. Therefore, we assume that also this difference is due to a sequencing error in the Reference sequence. The change from T to C at this position results in an amino acid substitution from Tryptophan (Y) to Cysteine (C).”

TABLE 1
Differences in nucleotide sequence between
rgMutHu and the MutHu reference sequence

| | | MutHu | | |
|---|---|---|---|---|
| Position | Gene | Ref | rgMutHu | remark |
| 1318 | NP | G | A | silent |
| 2339 | P | A | G | silent; also, G in pCVI-P^{MutHu} |
| 5123 | F | G | T > G | repaired |
| 8721-8727 | L | | 5 × A insert | repaired |
| 10383 | L | C | A > C | repaired |
| 10417 | L | G | A > G | repaired |
| 11749 | L | del 1 nt | A | error in Ref seq |
| 13529 | L | T | C | Y > C; also, C in pCVI-L^{MutHu} |

When comparing the nucleotide sequences of strains MutHu and MTH68 with those of other NDV strains, including strain Mukteswar (a mesogenic strain from which MTH68 is probably derived; Lancaster, 1964, Vet. Bull. 34:57-67), we noted a few differences in the last 20 nucleotides of the 5′-end of the viral genome (Appendix 2). These differences seem to be unique for MTH68/MutHu. Whether these differences are relevant for the oncolytic properties of these viruses is not clear.

2.4 Rescue of Infectious Virus from pFL-NDV MutHu

In order to generate infectious virus, we used the co-transfection system described above (and illustrated in FIG. 1) to transfect QM5 cells (derived from Quail) using plasmid pFL-NDV_MutHu and the helper plasmids pCVI-NP^{MutHu}, pCVI-P^{MutHu} and pCVI-L^{MutHu}. Rescue of infectious virus was successful as demonstrated by the presence of infectious virus in the allantoic fluid of ECE that were inoculated with the transfection supernatant (data not shown). The identity of the rescued virus was determined by RT-PCR followed by sequencing. The rescued virus was designated rgMutHu.

2.5 Restoration of Amino Acid Substitutions in the HN and M Proteins and Rescue of Corresponding Viruses

The observation that NDV-MutHu replicates in HeLa cells to virus titers that are at least 10-fold higher than those of strain MTH68 suggests that either one or both of the amino acid differences between these two strains is responsible for this phenotype. In order to address this issue, plasmid pFL-NDV_MutHu was used to restore the two amino acid mutations either individually or simultaneously to the MTH68 sequence. Using In-Fusion® or classical restriction-enzyme based cloning procedures, three different plasmids were generated containing the desired alterations. These plasmids were designated pFL-NDV_MutHu(M^{W165G}), pFL-NDV_MutHu(HN^{L277F}) and pFL-NDV_MutHu(M^{W165G}/HN^{L277F}), respectively. Subsequently, virus was rescued from these plasmids using the above described procedure. All three viruses were rescued successfully (Table 3). It should be noted that the DNA sequence of plasmid pFL-NDV_MutHu(M^{W165G}/HN^{L277F}) is identical to that of strain MTH68 and therefore the rescued virus rgMutHu(M^{W165G}/HN^{L277F}) can be regarded as rgMTH68.

TABLE 2
rescued viruses

| Virus | Remark |
|---|---|
| rgMutHu | 2 amino acid substitutions compared to |
| | MTH68 |
| rgMutHu(M^{W165G}) | Amino acid 165 in M restored |
| | from W to G |
| rgMutHu(HN^{L277F}) | Amino acid 277 in HN restored |
| | from L to F |
| rgMutHu(M^{W165G}/HN^{L277F}) = | Both amino acids restored to MTH68 |
| rgMTH68 | sequence |

W = Tryptophan;
G = Glycine;
L = Leucine;
F = Phenylalanine

### Example 3. Identify Whether One or Both of the Amino Acid Substitutions in NDV-MutHu are Responsible for the Difference in Growth Kinetics Between NDV-MutHu and the Parent Strain MTH68

3.1 Growth Kinetics in HeLa Cells

The rescued rg-viruses (Table 2) as well as the original MutHu and MTH68 viruses were used to determine their growth-kinetics in HeLa cells. Briefly, 4×10⁶ HeLa cells were seeded in 25 cm² cell culture flasks and grown overnight. The cells were infected using a MOI of 0.01 (i.e., 1 infectious virus particle per 100 cells), and at 8, 24 and 48 hours after infection the virus titer in the supernatant was determined by end-point titration on QM5 cells.

The data (FIG. 3) indicate that strains MutHu and rgMutHu yield at least 10-fold higher virus titers than MTH68. Furthermore, the data indicate that the mutation at amino acid position 277 in the HN gene is responsible for this effect. The M mutation does not seem to have an effect. This can be best seen when looking at the virus titers 24 h after infection, or even better when comparing the increase in virus titer between 8 h and 24 h (the exponential growth phase). The virus titer shows an increase of 3.5 (log 10) for MutHu, rgMutHu and rgMutHu(M^{W165G}), whereas this is 2.5 for MTH68, 2.7 for rgMutHu(HN^{L277F}) and 3.0 for rgMTH68 (Table 3).

TABLE 3
Virus titers (log10 TCID50/ml)

| | Time after infection (h) | | | |
|---|---|---|---|---|
| Virus | 0 | 8 | 24 | 48 |
| MTH68 | 4.8 | 4.5 | 7.0 | 7.0 |
| MutHu | 5.0 | 4.3 | 7.8 | 8.3 |
| rgMTH68 | 5.0 | 4.0 | 7.0 | 7.5 |
| rgMutHu(HN^{L277F}) | 5.0 | 4.3 | 7.0 | 7.3 |
| rgMutHu(M^{W165G}) | 4.8 | 4.3 | 7.8 | 7.5 |
| rgMutHu | 5.3 | 4.5 | 8.0 | 8.0 |

3.2 Time of Induction of Cytopathogenic Effect (CPE)

In an independent earlier experiment, we compared the growth-kinetics of strains MTH68, MutHu, and rgMutHu in HeLa cells using the same conditions as described above, except that also an infection with an MOI=1 was used. Also in this case we noted that strains MutHu and rgMutHu yield at least 10-fold higher virus titers compared to MTH68 (FIG. 4).

During the course of this experiment we noted differences in the onset of cytopathogenic effect (CPE) caused by the different viruses. Notably, MTH68 induced CPE earlier than either MutHu or rgMutHu (FIG. 5).

3.3 Conclusions
A reverse genetics system was established for the oncolytic NDV strain NDV-MutHu
Infectious virus, designated rgMutHu, was successfully rescued from cloned full-length NDV-MutHu cDNA
NDV-MutHu and rgMutHu have similar growth kinetics in HeLa cells
The HN²⁷⁷ mutation appears to be responsible for the improved growth kinetics of NDV-MutHu compared to MTH68
rgMutHu in which the HN and M mutations have been restored [rgMutHu(M^{W165G}/HN^{L277F})] is identical to rgMTH68
In HeLa cells MTH68 appears to induce CPE earlier than NDV-MutHu and rgMutHu, but NDV-MutHu and rgMutHu reach ˜10-fold higher virus titers
Genetic modification (including the insertion of therapeutic transgenes) of oncolytic NDV strains NDV-MutHu and MTH68 is now possible

### Example 4. Generation of Recombinant NDV-MutHu Strains with Enhanced Oncolytic and Immune Stimulating Properties Due to the Expression of Different Therapeutic Proteins

To this end, three different rgMutHu strains were generated, expressing the genes for:

1) Apoptin

2) B18R

3) Nivolumab

Apoptin (VP3 from chicken anemia virus) has been shown to selectively induce apoptosis in human tumor cells, but not in normal human cells.

B18R (from Vaccinia virus) is a homolog of the human IFN-β receptor. The secreted form of B18R acts as a decoy for IFN-β, thereby inhibiting IFN-mediated activation by cell signaling of the antiviral host response in naive cells.

Nivolumab is a human IgG4 anti-PD-1 monoclonal antibody (MAb) that is a checkpoint inhibitor, blocking a signal that prevents activated T cells from attacking the cancer, thus allowing the immune system to clear the cancer. Binding of Nivolumab to PD-1 on T-cells results in the elimination of downregulation of T-cell effector responses by cancer cells.

The properties of the recombinant viruses in comparison to the parent strain rgNDV-MutHu and strain MTH68 were examined by performing growth kinetics experiments and cytotoxicity assays in HeLa cells. In addition, we followed the fate of infected cells by using a RTCA that determines changes in attachment, size and morphology.

4.1 Generation of Recombinant Viruses

Recombinant NDV-MutHu viruses (rgNDV-MutHu) expressing Apoptin, B18R or Nivolumab were generated by means of the previously established reverse genetics system described above. Synthetic genes containing the open reading frames encoding the different proteins were obtained from GenScript Inc. and cloned between the P and M genes of NDV-MutHu (FIG. 6). The genes were provided with the necessary NDV gene-start and gene-end sequences in order to allow transcription by the vRNA polymerase.

Nivulomab as a MAb consists of two proteins, i.e., IgG heavy (H) and light (L) chains. In order to allow expression of both proteins from one gene, we generated a fusion-construct that encodes H and L chains as a fusion protein separated by a furin cleavage site and the T2A peptide (Chng et al., 2015, mAbs 7:403-412). Due to a ribosomal stop-start event at the T2A peptide during translation, this results in two separate proteins (H chain and L chain) in equimolar amounts. Furthermore, we used the H7 and L2 signal sequences for the H and L chain, respectively, to allow secretion of the proteins (Haryadi et al., 2015, PLoS ONE 10(2): e0116878. doi: 10.1371/journal.pone.0116878). The secreted H and L chains assemble with each other to form the final IgG molecule.

Infectious virus was rescued for all three constructs, and virus stocks were prepared by two passages in HeLa cells. The nucleotide sequences of the inserted genes in the different recombinant viruses were verified by means of nucleotide sequence analysis and found to be correct. Faithfull expression of Apoptin by rgMutHu-apoptin was verified by means of immunological staining of rgMutHu-apoptin infected monolayers using a MAb against Apoptin (FIG. 7). Expression of Nivolumab was verified and quantified by means of a commercial human IgG4 ELISA. Expression levels of Nivolumab reached approximately 2 μg/mL (FIG. 8).

4.2 Cytotoxicity Assays and Growth Kinetics

In order to examine the cytotoxic properties of the recombinant viruses and compare them to those of the parent virus rgMutHu as well as rgMTH68, we performed RTCA using a iCELLigence device which measures differences in cellular impedance. The functional unit of a cellular impedance assay is a set of gold microelectrodes fused to the bottom surface of a cell culture plate well. When submersed in an electrically conductive solution (such as buffer or standard tissue culture medium), the application of an electric potential across these electrodes causes electrons to exit the negative terminal, pass through bulk solution, and then deposit onto the positive terminal to complete the circuit. Because this phenomenon is dependent upon the electrodes interacting with bulk solution, the presence of adherent cells at the electrode-solution interface impedes electron flow. The magnitude of this impedance is dependent on the number of cells, the size and shape of the cells, and the cell-substrate attachment quality. Importantly, neither the gold microelectrode surfaces nor the applied electric potential has an effect on cell health or behavior (https://www.aceabio.com/product/icelligence/).

HeLa cell were grown in the wells of the device and after approx. 24 h the cells were infected with the different NDV strains (Table 4). The effect on cell proliferation, morphology change, and attachment was then monitored over a period of 4 or 5 days.

TABLE 4
NDV strains used.

| Strain | Remark |
|---|---|
| MTH68 | Original MTH68 strain |
| MutHu | Original MutHu strain |
| rgMTH68 | MTH68 generated by reverse genetics |
| rgMutHu | MutHu generated by reverse genetics |
| rgMutHu-Apoptin | rgMutHu containing the Apoptin gene |
| rgMutHu- | rgMutHu containing the Nivolumab |
| Nivolumab | gene |
| rgMutHu-B18R | rgMutHu containing the B18R gene |

FIGS. 9 and 10 show the results of the RTCA using HeLa cells infected with the different NDV strains at 24 h after seeding using a MOI of 0.01 and 0.1, respectively. The first noticeable observation is that the signal generated by all (MOI=0.01) or some (MOI=0.1) of the infected cultures increases above the value for non-infected HeLa cells. This increase does not correspond to cell proliferation (cell division) but is due to a change in morphology and size in the virus-infected cell cultures compared to non-infected cultures. This effect is reversed later in the infection cycle when cells start to die due to the cytotoxic effect of the viral infection. Thus, the rise and fall of the signal after virus infection is a combined effect of morphological changes and cell killing. The infection of HeLa cells induces cellular defense mechanisms (“danger signals”) resulting in the release of cytokines that stimulate the proliferation and/or morphology change of infected as well as non-infected cells. This seems to be in agreement with the observation that the effect is larger and longer-lasting at MOI of 0.01.

The difference in curves for the different viruses (best seen in FIG. 10; MOI=0.1) shows that the MTH68 strains are more cytotoxic than the MutHu strains. The cytotoxicity of the rgMutHu strains that carry an extra gene (Apoptin, B18R or Nivolumab) seems to be even further delayed.

We next studied the correlation between the RTCA results on the one hand and cell-killing & virus replication on the other. To this end, we determined the cell viability (percentage of living cells) and the virus titer at different time points after infection (MOI=0.1). Cell viability was determined by using the trypan-blue dye-exclusion assay, whereas infectious virus titers in the supernatant were determined by end point dilution on QM5 cells.

FIG. 11 shows that MTH68 and MutHu strains are more cytotoxic than the rgMutHu strains that carry an extra gene (Apoptin, Nivolumab or B18R). This appears to be in agreement with the RTCA results although the cytotoxicity seems to be underestimated in the RTCA compared to the viability assay, apparently due the above noted effect of virus infection on the morphology and size of the cells.

Analysis of the virus replication kinetics (FIG. 12) showed that final virus titers for MTH68 and rgMTH68 were approx. one log lower than those of MutHu and rgMutHu, confirming previous observations. Notably, strains rgMutHu-B18R and rgMutHu-Nivolumab reached virus titers at 72 h post infection that even exceeded those of the parent strain rgMutHu. This was not the case for rgMutHu-Apoptin. At 24 h post infection, however, the virus titers for rgMutHu-B18R and rgMutHu-Nivolumab (as well as rgNutHu-Apoptin) were significantly lower than those of rgMutHu, showing that replication was delayed, probably as a result of the larger genome size.

The insert-size of rgMutHu-Nivolumab is twice as large as that of rgMutHu-B18R and more than 4 times as large as that of rgMutHu-Apoptin. This difference in size seems to correlate with the cytotoxicity values at 72 h post infection. However, the RTCA signal for the rgMutHu-B18R is much lower compared to that of the other two strains (which are almost similar). This shows that other effects than the genome size are responsible for the observed difference in RTCA signal. These effects are most probably related to the biological functions of the therapeutic proteins.

In this example 4 we have generated three recombinant NDV-MutHu strains, each expressing a different therapeutic protein, i.e., Apoptin, B18R and Nivolumab. These proteins were chosen because expression of these proteins is expected to result in an enhancement of the oncolytic and/or immune-stimulating properties of NDV-MutHu. Our cytotoxicity assays suggest that the insertion of an extra gene results in a delay in the onset of cell killing. However, this does not necessarily result in lower virus titers. In fact, rgMutHu-B18R and rgMutHu-Nivolumab reached final virus titers which were higher than those of the parent strain rgMutHu (FIG. 12). It seems, therefore, that there is a balance between the onset and duration of cell-killing and the amount of progeny virus that is produced. In this respect, it is noteworthy that rgMutHu-Apoptin did not reach the same virus titers as the other two recombinant viruses. This suggests that expression of Apoptin by rgMutHu-Apoptin results in earlier cell-killing as would be expected from a pro-apoptotic protein. This seems to be confirmed by the cytotoxicity assays (FIG. 11).

Our data show that the results of the RTCA should be interpreted with some caution since the signal generated by the iCELLigence device underestimates the actual cytotoxic capacity of the recombinant MutHu strains. This is primarily caused by the effect on the RTCA signal of changes in cell morphology and cell size after infection. Thus, the RTCA signal shows a combined effect of morphological changes and cell killing.

The observation that the recombinant rgMutHu strains have a slower in vitro cytotoxicity (direct cell killing effect) in HeLa cells compared to the parent strain does not necessarily mean that they are inferior as oncolytic viruses for tumor treatment. It has been shown that the anti-cancer effects of replication-competent oncolytic viruses such as NDV are for a large part due to their immune-stimulating properties. Indeed, it has been shown that a non-pathogenic NDV is a potent inducer of type-I IFN and Dendritic Cell maturation, and that intra-tumoral injection of NDV results in distant tumor immune infiltration in the absence of distant virus spread (Zamarin et al., 2014, Sci. Transl. Med. 6(226 Whereas the presence of a transgene seems to have a negative effect on the rate of virus replication and cytotoxicity when compared to the parent strain, the ultimate virus titers of the transgene-expressing rgMutHu strains are comparable or even higher than those of the parent strain (FIG. 12). A slower replication rate in association with the production of a therapeutic protein will benefit the induction of immunological anti-tumor responses perhaps at a modest cost of cytotoxicity. However, compared to rgNDV strains that do not carry the F277L mutation, the intrinsic higher replication rate of rgMutHu strain will compensate for the loss of in vivo replication experienced by rgNDV strains. Therefore, the F277L mutation will enhance both the immune-stimulating as well as the cytotoxic effect.

An in-vitro effect was anticipated for rgMutHu-B18R since B18R is able to capture IFN-β thereby interfering with IFN-signaling. However, published data suggest that IFN-β expression by HeLa cells is poorly inducible, and that HeLa cells do not produce IFN-β after infection with NDV (Enoch et al., 1986, PLoS ONE 10(2): e0116878 Blach-Olszewska et al., 1977, Arch Immunol Ther Exp (Warsz) 25:683-91). This may explain why we did not observe a clear effect of B18R in rgMutHu-B18R infected HeLa cells. A biological effect is expected for rgMutHu-Nivolumab only in the context of in vivo application since the antibody exerts its function by binding to T-cells expressing PD-1.

### Example 5: A Method for Preparing an rgNDV Having Improved Replication in a Cancer Cell Over a Parent NDV and the Resulting rgNDV

The observation that the F to L substitution at amino acid position 277 in the HN protein of NDV results in improved replication in cancer cells indicates that the HN protein fulfills an as-yet not understood but important function in determining the extent of virus replication in these cells. The substitution of F at position 277 by an amino acid other than L might increase replication to even higher levels. Furthermore, amino acid substitutions at positions other than 277 in the HN protein may have similar or improved effects on the level of virus replication in cancer cells. Also, the combination of two or more amino acid substitutions at different positions in HN, or the deletion of one or more amino acids at specific positions in HN, may result in increased replication levels of rgNDV in cancer cells. Therefore, a systematic survey of the effect of amino acid substitutions and/or deletions in HN of rgNDV on replication in cancer cells is provided herein.

An rgNDV having improved replication in cancer cells over its parent NDV is obtained by generating rgNDV containing a genetically modified HN gene. Having identified L277 as an important mutation that affects virus replication, it is now provided by such techniques to perform studies to evaluate the effect of substitutions with other amino acids at this position or at other positions in the neighborhood of 277 or at positions elsewhere in HN. Additional studies may also focus on the identification of potential differences in the cellular interaction partners of wt and mutant HN. Using methods known in the art, specific mutations (i.e., substitution of a specific nucleotide by a different nucleotide) or deletions are introduced in a nucleic acid construct containing (part of) the nucleotide sequence encoding the HN protein of NDV. Based on the position(s) and type(s) of the nucleotide substitution(s) or deletions this will result in (a) specific amino acid substitution(s) or deletions in the HN protein. The genetically modified nucleic acid is used to replace the corresponding nucleotides in the full-length NDV genome, which can subsequently be used to generate infectious virus. This collection of mutant rgNDV can be used to examine replication in cancer cells, thus allowing the identification rgNDV with improved replication properties over the parent NDV.

### REFERENCES

Blach-Olszewska et al., (1977) Why HeLa cells do not produce interferon? Arch Immunol Ther Exp (Warsz) 25:683-91.
Chng et al., (2015) Cleavage efficient 2A peptides for high level monoclonal antibody expression in CHO cells, mAbs, 7:2, 403-412; http://dx.doi.org/10.1080/19420862.2015.1008351.
Enoch et al., (1986) Activation of the Human beta-Interferon Gene Requires an Interferon-Inducible Factor, Mol. Cell. Biol. 6:801-10.
Haryadi et al., (2015) Optimization of Heavy Chain and Light Chain Signal Peptides for High Level Expression of Therapeutic Antibodies in CHO Cells. PLoS ONE 10(2): e0116878. doi:10.1371/journal.pone.0116878.
Schirrmacher, (2015) Oncolytic Newcastle disease virus as a prospective anti-cancer therapy. A biologic agent with potential to break therapy resistance. Expert Opin. Biol. Ther. 15:17 57-71
Zamarin et al., (2014) Localized oncolytic virotherapy overcomes systemic tumor resistance to immune checkpoint blockade immunotherapy. Sci. Transl. Med. 6(226).
Zamarin & Palese, (2017) Oncolytic Newcastle Disease Virus for cancer therapy: old challenges and new directions. Future Microbiol. 7: 347-67.

APPENDIX 1
primers used for the generation of cDNA fragments and helper-plasmids
cDNA fragments

| Fragment | Size | Primer | Sequence (5′-3′) |
|---|---|---|---|
| C1 | 3.6 kb | Noss-9F | ACGACTCACTATAGGACCAAACAGAGAATCCGTGAG |
| | | (SEQ ID | |
| | | No. 5) | |
| | | Noss- | CCGGGAAGATCCAGGGCACTCTTCTTGCATGTTAC |
| | | 121R (SEQ | |
| | | ID No. 6) | |
| C2 | 3.7 kb | Noss-122F | GGGCCTGCCTCACTATGGTGGTAACATGCAAGAAG |
| | | (SEQ ID | |
| | | No. 7) | |
| | | Noss- | TGCATGTTACCACCAATGTGTCATTGTATCGCTTG |
| | | 123R (SEQ | |
| | | ID No. 8) | |
| C3 | 5.7 kb | Noss-125F | CAAGAAGGGAGATACGTAATATACAAGCGATACAATG |
| | | (SEQ ID | |
| | | No. 9) | |
| | | Noss- | TCGCTTGTATATTACTTGTTGTAGCAAAGAGCACC |
| | | 126R (SEQ | |
| | | ID No. 10) | |
| C8 | 2.0 | Noss-133 | GGCCTGGATCTTCCCATTATGCTGTCTGTATACGGTGC |
| | | (SEQ ID | |
| | | No. 11) | |
| | | Noss-10R | ATGCCATGCCGACCCACCAAACAAAGACTTGGTGAATG |
| | | (SEQ ID | |
| | | No. 12) | |
| RT-primer | | RT-21 | ACCAAACAGAGAATCCGTG |
| (C1, C2) | | (SEQ ID | |
| | | No. 13) | |
| RT-primer | | RT-45 | AGTCTTCAGTCATGGACAGC |
| (C3, C8) | | (SEQ ID | |
| | | No. 14) | |

Helper-plasmids (generated by In-Fusion ® cloning in pCVI)

| Gene | primer | sequence |
|---|---|---|
| NP | Noss-22F | CTCTAGAGTCGACCCTTCTGCCAACATGTCTTCCG |
| | (SEQ ID | |
| | No. 15) | |
| | Noss-23R | GGGAAGCGGCCGCCCGTCGGTCAGTATCCCCAGTC |
| | (SEQ ID | |
| | No. 16) | |
| P | Noss-24F | CTCTAGAGTCGACCCCAGAGTGAAGATGGCCACCTTC |
| | (SEQ ID | |
| | No. 17) | |
| | Noss- | GGGAAGCGGCCGCCCAGTGATCAGCCATTCAGCGC |
| | 25R (SEQ | |
| | ID No. 18) | |
| L | Noss-26F | CTCTAGAGTCGACCCGGGTAGGACATGGCGGGCTC |
| | (SEQ ID | |
| | No. 19) | |
| | Noss-27R | GGGAAGCGGCCGCCCTGCCTTTAAGAGTCACAGTTAC |
| | (SEQ ID | |
| | No. 20) | |

Sequences:
SEQ ID No. 1: Nucleic acid (cDNA) sequence of Newcastle disease virus strain (NDV) MutHu genome;
SEQ ID No. 2: Nucleic acid (cDNA) sequence of Newcastle disease virus strain rgMutHu-Apoptin genome created by reverse genetics as disclosed herein, wherein the nucleic acid additionally encodes the pro-apoptotic protein apoptin;
SEQ ID No. 3: Nucleic acid (cDNA) sequence of Newcastle disease virus strain rgMutHu-B18 genome created by reverse genetics as disclosed herein, wherein the nucleic acid additionally encodes a homolog of the human IFN-β receptor;
SEQ ID No. 4: Nucleic acid (cDNA) sequence of Newcastle disease virus strain rgMutHu-Nivolumab genome created by reverse genetics as disclosed herein, wherein the nucleic acid additionally encodes human IgG4 anti-PD-1 monoclonal antibody;
SEQ ID No. 21: Nucleic acid (cDNA) sequence of Newcastle disease virus strain 5′-UTR Mukteswar EF201805
SEQ ID No. 22: Nucleic acid (cDNA) sequence of Newcastle disease virus strain 5′-UTR NDV MuHu (Noss)
SEQ ID No. 23: Nucleic acid (cDNA) sequence of Newcastle disease virus strain 5′-end MTH68 (CVI)
SEQ ID No. 24: Nucleic acid (cDNA) sequence of Newcastle disease virus strain 5′-end MuHu (CVI)
SEQ ID No. 25: Consensus sequence 1 of SEQ ID No. 21 to 24

1. A Newcastle Disease Virus (NDV) for use in ontological treatment of a subject considered in need thereof, wherein said NDV having a mutation in the HN gene providing it with an increased replication capability in a human cancer cell, allowing a replication of said oncolytic NDV in a human cancer cell in higher levels than the parent strain of said NDV not having said mutation.

2. An NDV according to claim 1, wherein said parent strain comprises or is strain MTH-68/H and/or wherein the mutation in the HN gene leads to a change in amino acid sequence of hemagglutinin-neuraminidase, preferably wherein an amino acid in position 277, particularly phenylalanine (F), is substituted, more preferably wherein the amino acid in position 277 is substituted to an amino acid with a hydrophobic side chain except phenylalanine, more particularly wherein the amino acid in position 277 is substituted to leucine (L) at position 277 of the HN gene.

3. An NDV according to claim 1, wherein said subject is a mammal, particularly a mammalian animal or a human, subject.

4. A nucleic acid encoding a NDV, particularly a rgNDV, having a mutation in the HN gene, said mutation allowing replication of said NDV in a cancer cell to a higher level than replication of an otherwise identical NDV not having said mutation in the HN gene, preferably wherein the nucleic acid comprises or consists of at least one of SEQ ID No. 1 to No. 4 or parts thereof, preferably having a sequence identity of at least 75%, particularly at least 90%, more particularly 95%, preferably wherein the nucleic acid is derived from a NDV according to any one of the preceding claims.

5. A nucleic acid according to claim 4 wherein said mutated HN gene encodes hemagglutinin-neuraminidase with an amino acid substitution at position 277 to an amino acid with a hydrophobic side chain other than phenylalanine, particularly a leucine (L), more particularly wherein the mutated HN gene encodes HN^{F277L}.

6. A nucleic acid according to claim 4, additionally comprising a transgenic construct, wherein said transgenic construct provides for improved lysis of a cancer cell and/or increased replication capability in a human cancer cell, particularly improved lysis of a neoplastic tissue cell, preferably wherein said transgenic construct is selected from the group of nucleic acid constructs encoding one protein or a combination of two or more proteins and/or at least a part thereof selected from the group consisting of a protein that reduces or inhibits interferon expression, a protein with cytokine activity, a protein with antibody activity, a protein with apoptotic activity, a protein capable of blocking checkpoint inhibition, a green fluorescent protein (GFP), and/or a protein capable of enhanced binding (targeting) to a cancer cell.

7. A nucleic acid according to claim 4, comprising a or said transgenic construct, wherein the transgenic construct encodes a protein and/or a part thereof that reduces and/or inhibits interferon expression, such as an IFN-beta receptor, preferably viral protein B18R from vaccinia virus, particularly wherein the nucleic acid comprises or consists of the SEQ ID No. 3 or parts thereof, preferably having a sequence identity of at least 75%, particularly at least 90%, more particularly 95%.

8. A nucleic acid according to claim 4, comprising a or said transgenic construct, wherein the transgenic construct encodes a protein and/or a part thereof with apoptotic activity, such as viral protein-3 from chicken anemia virus, apoptin, particularly wherein the nucleic acid comprises or consists of the SEQ ID No. 2 or parts thereof, preferably having a sequence identity of at least 75%, particularly at least 90%, more particularly 95%.

9. A nucleic acid according to claim 4, comprising a or said transgenic construct, wherein the transgenic construct encodes an antibody and/or a part thereof capable of blocking checkpoint inhibition, such as an antibody directed against checkpoint inhibition protein PD-1, particularly wherein the nucleic acid comprises or consists of the SEQ ID No. 4 or parts thereof, preferably having a sequence identity of at least 75%, particularly at least 90%, more particularly 95%.

10. A nucleic acid according to claim 4, comprising a or said transgenic construct, wherein the transgenic construct encodes a protein and/or a part thereof capable of providing enhanced binding (targeting) of NDV to a cancer cell, particularly like one protein or a combination of two or more proteins and/or at least a part thereof selected from the group consisting of a modified HN protein that is fused to a single-chain antibody against a tumor-associated cell surface protein, a modified HN protein that is fused to a ligand that specifically binds to a tumor-associated cell-surface protein, and/or a bi-specific protein consisting of a single-chain antibody against the rgNDV HN protein fused to a single-chain antibody and/or a ligand that specifically binds to a tumor-associated cell-surface protein.

11. A nucleic acid, encoding a NDV, particularly a rgNDV, more particularly the nucleic acid according to claim 4, and/or having a mutation in the F gene, wherein said mutation is capable of improving oncolytic potential of said NDV, preferably wherein said mutated F gene encodes a fusion protein with an amino acid substitution at position 289 to an amino acid with a hydrophobic side chain other than leucine, particularly an alanine, more particularly wherein the mutated F gene encodes F^{L289A}.

12. A nucleic acid according to claim 4 additionally having a mutation in the RNA-editing sequence of the P gene that abolishes and/or decreases the expression of the V protein and/or additionally having a deletion in the NP-gene and/or in the V-gene and/or in the HN-gene.

13. A method for preparing a rgNDV, having improved replication in a cancer cell over a parent NDV, said method comprising the steps:
a. providing a nucleic acid construct encoding a HN gene with a mutation, wherein the mutation in the HN gene leads to a change in the expression of hemagglutinin-neuraminidase, wherein the amino acid, particularly phenylalanine (F), in position 277 is substituted, particularly wherein the amino acid in position 277 is substituted to an amino acid with a hydrophobic side chain, more particularly wherein the amino acid in position 277 is substituted to having a leucine (L) at position 277 of the HN gene,
b. incorporating said nucleic acid construct with said mutation in a nucleic acid encoding a rgNDV, particularly to achieve a nucleic acid according to anyone of claims 4 to 12,
c. using said nucleic acid encoding a rgNDV to produce infectious rgNDV,
d. comparing the replication characteristics in cancer cells of said rgNDV with the replication characteristics of said parent NDV,
e. selecting said rgNDV for further use when is shows improved replication characteristics over said parent NDV.

14. A method according to claim 13, wherein the nucleic acid encoding a rgNDV of step b further comprises a transgenic construct, preferably wherein said transgenic construct encodes a protein which leads to an improved lysis of a cancer cell, particularly improved lysis of a neoplastic tissue cell, more preferably wherein said transgenic construct is one or a combination of two or more selected from the group of nucleic acid constructs encoding a protein that reduces or inhibits interferon expression, encoding a protein with cytokine activity, encoding a protein with antibody activity, encoding a protein with apoptotic activity, encoding a protein capable of blocking checkpoint inhibition, encoding a green fluorescent protein (GFP), and/or encoding a protein capable of enhanced binding (targeting) to a cancer cell.

15. A method according to claim 13, wherein the nucleic acid encoding a rgNDV of step b carries a mutation in the F gene, particularly wherein said mutation is capable of improving oncolytic potential of said rgNDV, preferably wherein said mutation in the F gene comprises or is a mutation which encodes a fusion protein having an amino acid substitution in position 289, particularly a L289A substitution.

16. A method according to claim 13, wherein the nucleic acid encoding a rgNDV of step b further carries a mutation in the RNA-editing sequence of the P gene, particularly that abolishes and/or decreases the expression of the V protein.

17. A pharmaceutical formulation comprising particles of NDV according claim 1, preferably for use in oncological treatment of a subject considered in need thereof, in particular for the treatment of one or more indications selected from the group of brain tumors, like glioblastoma and/or astrocytoma, leukemia, lymphoma, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose & throat (ENT) tumors, tongue tumors, skin tumors, like melanoma, neuroblastoma, mesothelioma, renal cell carcinoma, fibrosarcoma, pheochromocytoma, head and/or neck carcinoma.

18. An NDV according claim 1, wherein it is used for the treatment of one or more indications selected from the group of brain tumors, like glioblastoma and/or astrocytoma, leukemia, lymphoma, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose & throat (ENT) tumors, tongue tumors, skin tumors, like melanoma, neuroblastoma, mesothelioma, renal cell carcinoma, fibrosarcoma, pheochromocytoma, head and/or neck carcinoma.

20. An NDV according to claim 1, wherein a replication of said oncolytic NDV in a human cancer cell in at least 2-fold higher levels than the parent strain of said NDV not having said mutation is achieved.

21. An NDV according to claim 1, which is encoded by and/or which comprises at least one of the nucleic acids according to SEQ ID No. 1 to 4 or parts thereof or having a sequence identity of at least 75%, particularly at least 90%, more particularly 95%.

22. An NDV according to claim 1, wherein said mutated HN gene encodes hemagglutinin-neuraminidase with an amino acid substitution at position 277 to an amino acid with a hydrophobic side chain other than phenylalanine, particularly a leucine (L), more particularly wherein the mutated HN gene encodes HNF277L.
